(19) United States
(12) Patent Application Publication
(10) Pub. No.: US 2004/0253648 A1
(43) Pub. Date: Dec. 16, 2004

(54) Method for creating nuclear receptor activity modulating pharmaceuticals

(75) Inventors: Robert J. Fletterick, Hillsborough, CA (US); Sabine Borngraeber, San Francisco, CA (US); John D. Baxter, San Francisco, CA (US); Thomas S. Scanlan, San Francisco, CA (US); Grazia Chiellini, Madison, WI (US); Paul Webb, San Francisco, CA (US)

Correspondence Address: QUINE INTELLECTUAL PROPERTY LAW GROUP, P.C., P O BOX 458, ALAMEDA, CA, 94501, US

(73) Assignee: The Regents of the University of California, Oakland, CA

(21) Appl. No.: 10/732,901

(22) Filed: Dec. 9, 2003

Related U.S. Application Data
(63) Continuation-in-part of application No. 10/317,034, filed on Dec. 10, 2002.
(60) Provisional application No. 60/453,608, filed on Mar. 10, 2003.
(60) Provisional application No. 60/526,931, filed on Dec. 3, 2003.

(51) Int. Cl. G01N 33/53; G01N 33/567

(52) U.S. Cl. 435/7.2

(57) ABSTRACT

Methods for screening, identifying and/or designing agents that modulate nuclear receptors are provided. These agents contact a site on a nuclear receptor involved in dimer/heterodimer formation, cofactor molecule interactions, and/or folding, which is termed the nuclear receptor dimer/heterodimer regulatory site (DHRS). Methods employing the DHRS are included, along with nuclear receptor:agent complexes and libraries of agents.

### CROSS-REFERENCE TO RELATED APPLICATIONS

This application is related to U.S. patent applications U.S. Ser. No. 10/317,034 by Baxter et al. filed Dec. 10, 2002; U.S. Ser. No. 60/453,608 by Fletterick et al. filed Mar. 10, 2003; and Attorney Docket No. 4071-000510US by Fletterick et al. filed Dec. 3, 2003. The present application claims priority to, and benefit of, these applications, which are incorporated herein by reference in their entirety for all purposes.

### STATEMENT AS TO RIGHTS TO INVENTIONS MADE UNDER FEDERALLY SPONSORED RESEARCH AND DEVELOPMENT

The invention was made with United States Government support under grant (or contract) numbers DK53417, DK41842, DK52798 and DK058390, awarded by the National Institutes of Health. The United States Government may have certain rights in the invention.

### FIELD OF THE INVENTION

The present invention is in the field of nuclear receptor modulation. The invention also relates to agents that modulate nuclear receptors activity via a dimer/heterodimer regulatory site (DHRS), agent-nuclear receptor complexes, screening methods for agents that modulate nuclear receptor activity, libraries of agents that modulate nuclear receptor activity and methods of treating diseases with agents that modulate nuclear receptor activity.

### BACKGROUND OF THE INVENTION

Nuclear receptors represent a superfamily of proteins that specifically bind and are regulated by physiologically relevant small molecules, such as hormones, vitamins, fatty acids, and the like. Binding of the relevant small molecule(s) to a nuclear receptor induces the receptor to modulate transcription in the cell, in a positive or negative way; the receptor-ligand complex can have transcription independent actions as well. Unlike integral membrane receptors and membrane-associated receptors, nuclear receptors mostly reside in either the cytoplasm or nucleus of eukaryotic cells. Thus, nuclear receptors comprise a class of intracellular, soluble, ligand-regulated transcription factors.

The biology and physiology of several nuclear receptors have been ascertained in some detail. For example, the mechanism of thyroid hormone action is reviewed in Yen (2001) “Physiological and Molecular Basis of Thyroid Hormone Action” Physiological Reviews 81(3): 1097-1142, and the references cited therein. Known and well characterized nuclear receptors include those for glucocorticoids (GRs), androgens (ARs), mineralocorticoids (MRs), progestins (PRs), estrogens (ERs), thyroid hormones (TRs), vitamin D (VDRs), retinoids (RARs and RXRs), and the peroxisome proliferator activated receptors (PPARs) that bind eicosanoids. Many ligands have been identified for nuclear receptors. For example, cortisol is a native ligand for the glucocorticoid receptor, while 3,5,3′-triiodo-L-thyronine (also referred to as triiodothyronine, T₃, or “thyroid hormone”) is a native ligand for the thyroid hormone receptor.

The so called “orphan receptors” are also considered part of the nuclear receptor superfamily, because they are structurally homologous to classic nuclear receptors such as steroid and thyroid receptors. While ligands have not been identified for orphan receptors (hence the designation “orphan” receptors), it is likely that small molecule ligands exist and will be discovered in the near future for many of these putative of transcription factors. Generally, nuclear receptors specifically bind physiologically relevant small molecules with high affinity. Apparent Kd's are commonly in the 0.01-20 nM range, depending on the nuclear receptor/ligand pair.

Nuclear receptors are involved in myriad of physiological processes and medical conditions such as hypertension, heart failure, atherosclerosis, inflammation, immunomodulation, hormone dependent cancers (e.g., breast, thyroid, prostate cancer, ovarian cancer, bone cancer, etc.), modulation of reproductive organ function, hypothyroidism, hyperthyroidism, hypercholesterolemia, hyperlipidermia, and other abnormalities of lipoproteins, diabetes, osteoporosis, mood regulation, mentation, aldosteronism, Cushing's syndrome, hirsutism and obesity. Consequently, it is advantageous to develop molecules that modulate the activities of nuclear receptors, e.g., by inactivating the receptor, by activating the receptor, etc.

Certain progress has been made in this regard. For example, U.S. Pat. No. 5,883,294 by Scanlan et al. (“Selective Thyroid Hormone Analogues”) describes, e.g., several classes of artificial thyroid hormone receptor ligands. Similarly, U.S. Pat. No. 6,266,622 by Scanlan et al. (“Nuclear Receptor Ligands and Ligand Binding Domains”) also describes several classes of thyroid hormone receptor ligands. For example, superagonists are described in the '622 patent, in which, e.g., the interactions of the ligand with various receptor residues (e.g., Arg 262, Arg 266 and Arg 228) in the ligand binding pocket are optimized. However, more solutions are needed.

This invention provides methods and compositions of molecules that modulate nuclear receptor activation through the surprising discovery of a novel site on nuclear receptors that is involved in nuclear receptor dimerization/heterodimerization, binding of cofactor molecules and an appropriate folding of the C-terminal F-domain of the steroid receptors against the ligand binding domain. The site is termed the nuclear receptor dimer/heterodimer regulatory site (DHRS). This and many other features of the invention will become apparent upon review of the following.

### SUMMARY OF THE INVENTION

This invention derives, in part, from the surprising discovery of a new site on nuclear receptors that is involved in dimer/heterodimer formation and/or cofactor molecule interactions, and that a molecule that contacts this site blocked dimer/heterodimer formation of the nuclear receptor. This site is termed the nuclear receptor dimer/heterodimer regulatory site (DHRS). Thus, the invention provides methods for identifying and designing agents that modulate (modulators) nuclear receptors or other regulatory molecules that contact this site, nuclear receptor-agent (modulator) complexes, including crystal structures thereof, therapeutic methods and compositions and several associated features such as systems and kits. For example, agents (modulators) that preferentially bind to the DHRS are provided in the invention.

Accordingly, in a first aspect, the invention provides methods of screening for a test agent that modulates dimer/heterodimer formation of nuclear receptors, and/or modulates cofactor molecule (e.g., coactivator or corepressor) interactions or appropriate folding of nuclear receptors (and/or the agents produced by the methods). For example, the methods include contacting at least one nuclear receptor dimer/heterodimer regulatory site of at least one nuclear receptor with a test agent; and detecting a change in a level of dimer/heterodimer formation and/or detecting a change in cofactor molecule interactions of the at least one nuclear receptor that is mediated by the test agent, e.g., compared to a control. In one embodiment, the at least one nuclear receptor comprises at least two nuclear receptors (e.g., where one of the at least two nuclear receptors is a retinoid X receptor (RXR)). Any of above steps can be performed in vitro, or in vivo, or in any combination thereof. For example, steps of the method (or agents of the invention produced by binding of the agent to the receptor) can be in a cell-free in vitro system (e.g., a transcription/translation system), or in a cell, in a tissue, or in a mammal.

In further aspects, the change in the level of dimer/heterodimer formation of the at least one nuclear receptor can be compared to the level of dimer/heterodimer formation in a control, e.g., where the difference in the level of dimer/heterodimer formation in the contacted DHRS and the level in the control indicates that the agent alters dimer/heterodimer formation of the at least one nuclear receptor. In certain embodiments, the control is exposed to a lower concentration (or absence) of test agent.

In alternative embodiments, the change in the interactions of cofactor molecules with the at least one nuclear receptor can be compared to the interactions in a control, e.g., where the difference in the interactions in the contacted DHRS and the interactions in the control indicates that the agent alters or modulates the interactions of the at least one nuclear receptor with cofactor molecules. In certain embodiments, the control is exposed to a lower concentration of, or no, test agent.

In a related aspect, the invention provides methods of prescreening for an agent that modulates dimer/heterodimer formation or cofactor molecule interactions with a nuclear receptor. The methods include contacting a nuclear receptor dimer/heterodimer regulatory site with a test agent; and, detecting specific binding of the test agent to said regulatory site. In one embodiment, the specific binding indicates that the test agent is a candidate modulator of dimer/heterodimer formation or cofactor molecule interactions.

A test agent of the invention can be any of a variety of molecules. In some embodiments, the test agent can be a small organic molecule. In alternative embodiments, the test agent can be a peptide, e.g., less than 15 amino acids, less than 10 amino acids, less than 8 amino acids, etc. In certain embodiments, the peptide is unrestrained, while in other embodiments, the peptide can be cyclized or constrained. The peptide can be composed of natural, synthetic or a combination of natural and synthetic amino acids. In certain embodiments, the test agent is an agent other than antibody, a protein, or a nucleic acid. Typically, the test agent is contacted directly to the at least one DHRS. Optionally, the test agent is contacted to a cell containing the at least one regulatory site, or optionally, the test agent is contacted to an animal comprising a cell containing the at least one regulatory site.

In another closely related class of methods, methods of identifying one or more agents (modulators) for at least one nuclear receptor (and the agents identified by the methods) are provided. In the methods, a plurality of putative modulators are provided, the plurality of modulators are contacted to at least one nuclear receptor dimer/heterodimer regulatory site of a nuclear receptor, where at least one of the putative modulators binds the DHRS, and the putative modulators are tested for modulator activity on the nuclear receptor, thereby identifying the one or more modulators of the nuclear receptor. In one embodiment, the testing includes binding the plurality of putative modulators to the least one DHRS; selecting for members of the plurality of putative modulators that bind the at least one DHRS; and, testing the resulting bound nuclear receptor for modulator activity (e.g., nuclear receptor activation or repression of nuclear receptor activity). Any of these steps can be performed in vitro, or in vivo, or in any combination thereof.

In yet another related aspect, the invention provides methods of designing a compound to contact a nuclear receptor dimer/heterodimer regulatory site (DHRS) (and compounds designed by such methods). In the methods, a three dimensional model of a protein or polypeptide comprising the nuclear receptor dimer/heterodimer regulatory site (DHRS) is provided. Binding of one or more compounds to the three dimensional model is modeled, thereby identifying one or more compound that binds to the DHRS. In one embodiment, modeling binding includes using a computer program e.g., DOCK, Catalyst, MCSS/Hook and/or other computer programs known by those of skill in the art (free or commercially available), to design the putative compound that binds the DHRS.

This, in turn, provides methods of designing an agent that contacts a DHRS using information provided by a crystal structure (e.g., for rational compound design approaches using models that take the crystal structure information into account). For example, in the methods, an information set derived from the crystal structure of a thyroid hormone's DHRS bound to 3,5-dimethyl-4-(4′-hydroxy-3′-benzyl)benzyl-phenoxy acetic acid (GC-24) is accessed, and, based on information in the information set, a prediction is made regarding whether a putative compound will interact with one or more three dimensional features of a nuclear receptor, e.g., to provide a compound that contacts the DHRS (e.g., binding is modeled using any available modeling tool and the crystal structure of the invention). For example, the information set can include atomic coordinate information of Appendix 1, or graphical modeling of that data, e.g., as provided by the various figures herein. Similarly, systems that include an information storage module and an information set derived from a crystal structure of a thyroid hormone's DHRS bound to GC-24 are a feature of the invention. In a related aspect, crystals of nuclear hormone receptor DHRS (e.g., thyroid receptor's DHRS) and an agent, e.g., GC-24, are also a feature of the invention.

In addition to providing agents produced by any of the methods above (or any combination thereof), the invention also provides a nuclear receptor:modulator complex compositions that includes a nuclear receptor bound to an agent, where the agent preferentially binds a nuclear receptor dimer/heterodimer regulator site (DHRS) of the nuclear receptor. This complex can be identified by the methods above, or by any other method. The nuclear receptor modulator complex can be in vitro, or in vivo. In one embodiment, the complex is in a cell. The complex can also be in a mammal or a non-mammal. The nuclear receptor:agent complexes produced by the methods of the present invention optionally inhibit or reduce the function of the nuclear receptor, such as dimerization or heterodimerization, binding of one or more cofactor molecules, and/or appropriate folding of the ligand binding domain of the nuclear receptor.

Libraries comprising a plurality of different agents (modulators) produced by any of the methods herein are also a feature of the invention. For example, the invention provides libraries of modulators that specifically bind a nuclear receptor dimer/heterodimer regulator site (DHRS) of a nuclear receptor. The libraries can be formatted as modulator-nuclear receptor complexes, or as modulators. The libraries can be spatially organized (e.g., in a gridded array) or can exist in any other logically accessible format.

For any of the methods or compositions (including any agent (modulator), modulator-nuclear receptor complex, library thereof, or any other composition of the invention noted herein), an agent contacts a nuclear receptor dimer/heterodimer regulatory site (DHRS). Typically, the DHRS comprises a hydrophobic cluster and is located on the surface of the nuclear receptor. In certain embodiments, the DHRS can include a region comprising polar and/or non-polar amino acids proximal to the hydrophobic cluster. Optionally, a solvent-based or solvent accessible region is included in the DHRS.

The hydrophobic cluster representing the DHRS of a selected nuclear receptor can be ascertained, for example, by comparison to an identified DHRS of a similar nuclear receptor (as described herein). For example, the DHRS of a thyroid hormone receptor β comprises residues Valine 376, Leucine 400, Leucine 422, and Valine 425; in a similar nuclear receptor, the DHRS would include residues/positions corresponding to these amino acids. In certain embodiments, the DHRS of the thyroid hormone receptor P further includes residues Serine 381, Aspartate 382, Glutamate 393, Glutamate 396, and Arginine 429. In another example, the DHRS comprises residues Valine 322, Leucine 346, Leucine 368, and Valine 371 of a thyroid hormone receptor α. Other examples include, but are not limited to: the DHRS of a peroxisome proliferator activated α receptor comprising residues Alanine 381, Valine 405, Leucine 427, and Methionine 430; the DHRS of a peroxisome proliferator activated y receptor comprising residues Valine 390, Leucine 414, Leucine 436, and Methionine 439; the DHRS of a retinoic acid α receptor comprising residues Isoleucine 332, Leucine 356, Leucine 378, and Isoleucine 381; the DHRS of a pregnane X receptor comprising residues Isoleucine 346, Alanine 370, Methionine 394, and Leucine 397; the DHRS of a vitamin D receptor comprising residues Isoleucine 336, Serine 360, Isoleucine 384, and Leucine 387, the DHRS of an androgen receptor comprising Leucine 810, Isoleucine 835, Threonine 860, and Leucine 863; the DHRS of an estrogen receptor comprising Isoleucine 451, Threonine 483, Leucine 508, and Leucine 511; and a DHRS of a progesterone receptor comprising a Leucine 824, Isoleucine 849, Threonine 874, and Leucine 877.

An agent of the invention can be any of a variety of molecules. For example, an example agent is a small organic molecule. In alternative embodiments, the agent can be a peptide, e.g., less than 15 amino acids, less than 10 amino acids, less than 8 amino acids, etc. In certain embodiments, the peptide is unrestrained, while in other embodiments, the peptide can be cyclized or constrained. The peptide can be composed of natural, synthetic or a combination of natural and synthetic amino acids. In certain embodiments, the agent is an agent other than an antibody, a protein or a nucleic acid. In certain embodiments, the agent is contacted directly to the at least one DHRS or contacted to a cell containing the at least one DHRS or contacted to an animal comprising a cell containing the at least one DHRS, etc.

An example of an agent (modulator) identified or producible by the methods of the invention is GC-24 (of course, an agent of the invention is optionally an agent other than GC-24). Considerable structural information is provided herein regarding GC-24 and related compounds as modulators, including a crystal structure of GC-24 bound to the DHRS of a thyroid hormone receptor (TR).

For any of the methods or compositions of the invention, an agent (modulator) of the invention can modulate nuclear receptor activation by, e.g., inactivating the nuclear receptor (e.g., by repression) or activating the receptor (e.g., by disrupting or dissociating a repressor or corepressor or by allowing association of an activator or coactivator). Typically, an agent of the invention modulates nuclear receptor activation by inhibiting dimer/heterodimer formation of the nuclear receptor, e.g., where the change in the level of dimer/heterodimer formation correlates with an activation of the at least one nuclear receptor, or where the change in the level of dimer/heterodimer formation correlates with a repression of the at least one nuclear receptor activity. In one example, the agent masks residues in the DHRS and prevents dimer/heterodimer formation, thereby modulating nuclear receptor activation. In another example, the agent modulates interactions of at least one nuclear receptor and a cofactor molecule. In certain embodiments, the agent modulates nuclear receptor activation by inhibiting activation of activation function 1 (AF-1). Alternatively, the agent modulates nuclear receptor activation by activating activation function 1, (AF-1). In certain embodiments, the agent modulates gene transcription, e.g., of at least one nuclear receptor responsive gene. In certain embodiments, an agent of the invention modulates nuclear receptor activation by modulating the nuclear receptor conformation that, e.g., stabilizes or destabilizes, the bound ligand in the nuclear receptor. For example, an agent of the invention can modulate the off-on rate, e.g., increase off rate, increase on rate, decrease off rate, decrease on rate, of the ligand to the nuclear receptor compared to a control.

Generally, the modulator activity of an agent can be confirmed in any of the methods of the invention, or for any of the compositions of the invention by any of the a variety of methods, e.g., by detecting the level of dimer/heterodimer formation, by detecting cofactor molecule interactions, by binding of the agent to the DHRS and testing for modulation of nuclear receptor activation or repression, or by another appropriate activity assay, in vitro or in vivo (or a combination thereof). For example, the level of dimer/heterodimer formation and/or cofactor molecule interactions can be detected by detecting expression of at least one nuclear receptor responsive gene, or a nuclear receptor responsive element operably linked to a reporter gene. In another example, the level of dimer/heterodimer formation and/or cofactor molecule interactions is detected by a gel shift assay, a fluorescence assay, a chromatography assay, immunochemistry assays (e.g., immunoprecipitation, western assays, far western assays, etc.), fusion tags, two-hybrid systems, etc.

Any of a variety of nuclear receptors can be used in the methods and compositions of the present invention, including a thyroid hormone receptor, a β thyroid hormone receptor, an alpha thyroid hormone receptor, a glucocorticoid receptor, an estrogen receptor, an androgen receptor, a mineralocorticoid receptor, a progestin receptor, a vitamin D receptor, a retinoid receptor, a retinoid X receptor, a peroxisomal proliferator activated receptor, an estrogen-receptor related receptor, a short heterodimer partner, a constitutive androstane receptor, a liver X receptor (LXR), a pregnane X receptor, a HNF-4 receptor, a farnesoid X receptor (FXR) and an orphan receptor. Nuclear receptors can include nuclear receptors expressed by human and non-human species including vertebrates and invertebrates. A database of nuclear receptors is available on the World Wide Web at receptors.ucsf.edu/NR/multali/multali.html. The invention can utilize any isoform of the relevant receptors. This is particularly useful to target nuclear receptor isoform-specific diseases.

The nuclear receptor in the methods and compositions of the invention can be a liganded receptor or an unliganded receptor. For example, an agent of the invention can modulate nuclear receptor activation by inhibiting activation of a liganded or unliganded nuclear receptor. Alternatively, the agent can modulate nuclear receptor activation by activating a liganded or unliganded nuclear receptor.

The present invention also provides methods of treatment, e.g., using any the agents (modulators) of the invention, e.g., as identified by any of the methods above. For example, the invention provides methods of treating a subject having a disease state which is alleviated by treatment with a nuclear receptor modulator, in which a therapeutically effective amount of an agent of the invention is administered to the subject (e.g., a human, or, in a veterinary application, an animal such as a non-human mammal) in need treatment. In one typical class of embodiments, the agent (modulator) is mixed with one or more pharmaceutically acceptable excipients prior to administration.

Example of diseases that can be treated using the agents (modulators) of the invention include, but are not limited to: hyperthyroidism, aldosteronism, Cushing's syndrome, hirsutism, cancer, thyroid cancer, breast cancer, prostate cancer, bone cancer, ovarian cancer, hypercholesterolemia, hyperlipidemia, atherosclerosis, obesity, cardiac arrhythmia, modulation of reproductive organ function, hypothyroidism, osteoporosis, hypertension, glaucoma, inflammation, immunomodulation, diabetes, and/or depression.

Agents (modulators) of the invention can also be used for combination therapy. In certain embodiments, an agent of the invention is co-administered with an agonist or an antagonist a nuclear receptor. In one aspect of the invention, the co-administration of the agent and the agonist or the antagonist of the nuclear receptor counteracts at least one deleterious effect (e.g., an undesired or unintended consequence or side effect) of the agonist or the antagonist. For example, steroids with glucocorticoid activity are used extensively as immunosuppressant and anti-inflammatory agents. However, the benefits of this therapy are countered by deleterious effects of the steroids. Many of the beneficial effects do not require dimerization of the glucocorticoid receptor to be elicited, while many of the deleterious effects appear to require receptor dimer formation. Thus, the agent of the invention can be administered with a nuclear agonist, e.g., steroid with glucocorticoid activity, to selectively modulate the receptor's, e.g., the glucocorticoid receptor's, actions.

Systems are also provided in the invention. In one embodiment, the system includes a screening system for screening test agents that modulate dimer/heterodimer formation and/or cofactor molecule interactions of nuclear receptors. For example, the screening system includes at least one polypeptide (e.g., a full or partial nuclear receptor amino acid sequence), where the at least one polypeptide comprises a nuclear receptor dimer/heterodimer regulatory site (DHRS); and, instructions for detecting dimerization/heterodimerization or cofactor molecule interactions of the at least one polypeptide. In certain embodiments, the polypeptide is provided by a nucleic acid, which encodes the polypeptide.

The invention also provides a prescreening system for prescreening a test agent that bind to a nuclear receptor dimer/heterodimer regulator site (DHRS). The prescreening system includes a polypeptide that comprises the DHRS; and, instructions for detecting specific binding of the test agent to the DHRS.

A system for designing putative compounds that contact a nuclear receptor dimer/heterodimer regulatory site (DHRS) is also provided. For example, the system includes a three dimensional model of a protein or polypeptide comprising a nuclear receptor dimer/heterodimer regulatory site (DHRS). The system also typically includes features for user-interface with the model, and, e.g., instructions for modeling binding of one or more compounds to the three dimensional model to design at least one putative compound that contacts the DHRS.

Kits comprising any composition of the invention are also a feature of the invention. Kits typically comprise one or more composition of the invention, e.g., packaged in one or more containers. The kits optionally provide instructions, e.g., for practicing one or more method herein.

Definitions

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a cell” includes a combination of two or more cells, and the like.

Unless defined otherwise, all technical and scientific terms used herein have the same meaning as commonly understood by one of ordinary skill in the art to which the invention pertains. In describing and claiming the present invention, the following terminology will be used in accordance with the definitions set out below.

The phrase “nuclear receptor dimer/heterodimer regulatory site” or “DHRS” refers to a hydrophobic cluster of amino acids that are co-localized or otherwise positioned on the surface of a nuclear receptor. The DHRS is involved in dimerization/heterodimerization of nuclear receptors. This site is also involved in interactions of cofactor molecules with a nuclear receptor and folding, e.g., folding of the C-terminal F-domain of the steroid receptors against the ligand binding domain, of a nuclear receptor.

The term “hydrophobic cluster” refers to three or more amino acids that are spatially related to each other in a three-dimensional polypeptide. The amino acids do not have to be found sequentially in the polypeptide chain to be a part of the hydrophobic cluster, although they can be.

The term “surface of a nuclear receptor” refers to a location(s) that is part of a nuclear receptor molecule that is solvent accessible, e.g., reachable via agents (e.g., test agents, modulators, etc.) when the receptor is not bound to other species (e.g., dimer or heterodimers partners, corepressors, coactivators, other cofactor molecules, DNA and ligands, etc.).

The term “proximal” refers to a region of the DHRS that is situated near the hydrophobic cluster region of the DHRS. “Proximal” refers to the close spatial relationship between the regions.

The term “solvent-based region” refers to a solvent-accessible region of the DHRS that permits an agent (modulator) to contact the region. The solvent-based region can include water or other solvent molecules that interact with moieties of the agent, as well as receptor residues.

The terms “agent” and “modulator” are generally used interchangeably herein. The agent (or modulator) is a compound that, when bound to the DHRS of a nuclear receptor, affects, alters, regulates, controls, or otherwise “modulates” receptor dimer/heterodimer formation and/or the interaction between a nuclear receptor and a cofactor molecule(s), e.g., a coactivator or a corepressor. This modulation can lead to activating or inactivating a nuclear receptor, thereby activating or repressing gene function. In some cases, nuclear receptors can act through second messenger signaling pathways, and the invention would apply to these actions as well. A “putative modulator” is a test agent to be tested for modulator activity.

A “cofactor molecule” is a molecule, e.g., protein, nucleic acid, small molecule, etc., that binds to the nuclear receptor and modulates activity of the receptor. Examples of cofactor molecules include, but are not limited to, corepressors (or repressors), coactivators (or activators), etc. A cofactor molecule can also generally refer to a coregulatory molecule.

A “thyroid hormone receptor” is a protein that is the same as or is similar to a known thyroid hormone receptor, wherein the protein is activated by thyroid hormone. Typically, if the protein is similar to the known receptor, it is more similar to a known thyroid receptor than it is to another identified receptor type. Known receptors that are annotated as being members of a given family of receptors can be found in GenBank™ or other public databases, e.g., a database of nuclear receptors is available on the World Wide Web at, for example, receptors.ucsf.edu/NR/multali/multali.html. Similarly, a “glucocorticoid receptor” is a protein that is the same as or similar to a known glucocorticoid receptor, where the protein binds a glucocorticoid such as cortisol. In general, a given nuclear hormone receptor type is a protein that is the same as or similar to a given nuclear hormone receptor type that is activated by the relevant natural cognate ligand. In all cases, the receptor may be activated by other ligands as well. Indeed, because of this receptor-ligand cross-talk, it is not formally correct to identify a receptor based simply upon which hormone(s) it binds to—for example, the mineralocorticoid receptors bind cortisol (a glucocorticoid). Thus, a receptor is classified based upon its degree of similarity to a known receptor that has been identified as a given receptor type (and typically is named, at least initially, based upon its primary hormone binding activity) and upon whether the receptor is activated in response to a given hormone. In this context, the degree of similarity that can be used to identify the receptor is somewhat flexible—many receptors are homologous to one another, showing at least some degree of similarity. Typically, a receptor is fit into a given family of receptors (e.g., the family of thyroid receptors) based upon how closely similar it is to other members of the family as compared to other receptor families and upon its ligand specificity. One can group receptor families into branches of an evolutionary tree to show relationships between family members and/or between families. Many software programs are publicly available for performing sequence similarity comparisons, including BLAST, BESTFIT, FASTA and many others. For a review of available sequence alignment and clustering methods and tools, see also Durbin et al. (1998) Biological Sequence Analysis: Probabilistic Models of Proteins and Nucleic Acids Cambridge University Press; and Mount (2001) Bioinformatics Sequence and Genome Analysis Cold Spring Harbor Press.

A “nuclear receptor” is a receptor that activates or represses transcription of one or more genes in the nucleus (but can also have second messenger signaling actions), typically in conjunction with other transcription factors. The nuclear receptor is activated by the natural cognate ligand for the receptor. Nuclear receptors are ordinarily found in the cytoplasm or nucleus, rather than being membrane-bound.

Unless otherwise specified, “in vitro” implies that something takes place outside of an organism or cell. “In vivo” implies that it takes place inside of a cell; the cell can be in culture or in a tissue, or an organism, or the like.

A “nuclear receptor responsive gene” (NRRG) is a gene whose transcription is altered in a cell in response to a nuclear receptor. The receptor can modulate the activity of the gene in the absence of the nuclear ligand, or, in some embodiments, in response to second messenger signaling pathways. Activation or inactivation of the receptor can be modulated by binding of an agent, which causes the receptor to differ in its activation or repression of the gene. The receptor can act while bound to DNA or while bound to other proteins directly or indirectly involved in transcription of the gene. The activity of the nuclear receptor responsive gene can also be modulated through nuclear receptor effects on second messenger signaling pathways.

GC-24 is a compound having the formula:

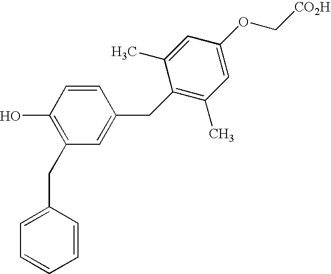

or a salt or ion thereof.

The term “test agent” refers to an agent (e.g., a putative modulator) that is to be screened in one or more of the assays described herein. The agent can be essentially any compound. It can exist as a single isolated compound or can be a member of a chemical (e.g., combinatorial) library.

A “library” is a set of compounds or compositions. It can take any of a variety of forms, e.g., comprising an intermingled or “pooled” set of compositions, or a set of compositions having spatial organization (e.g., an array, e.g., a gridded array), or logical organization (e.g., as existing in a database, e.g., that can locate compounds or compositions in an external storage system).

The term “database” refers to a system or other means for recording and retrieving information. In preferred embodiments, the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g., computer-based) databases, e.g., those used to track modulator activity (or putative modulators during the various screening processes herein). Computer systems for use in storage and manipulation of databases are well known to those of skill in the art and include, but are not limited to “personal computer systems”, mainframe systems, distributed nodes on an inter- or intra-net, data or databases stored in specialized hardware (e.g., in microchips), and the like.

A “therapeutically effective amount of an agent” is an amount of the modulator that is sufficient to provide a beneficial therapeutic effect, typically when administered over time.

The terms “nucleic acid” or “oligonucleotide” or grammatical equivalents herein refer to at least two nucleotides or analogs covalently linked together. A nucleic acid of the present invention is preferably single-stranded or double stranded and will generally contain phosphodiester bonds, although in some cases, as outlined below, nucleic acid analogs are included that can have alternate backbones, comprising, for example, phosphoramide (Beaucage et al. (1993) Tetrahedron 49(10): 1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 1419); phosphorothioate (Mag et al. (1991) Nucleic Acids Res. 19:1437; and U.S. Pat. No. 5,644,048 to Yau); phosphorodithioate (Briu et al. (1989) J. Am. Chem. Soc. 111:2321); O-methylphophoroamidite linkages (see Eckstein, Oligonucleotides and Analogues: A Practical Approach, Oxford University Press); and peptide nucleic acid backbones and linkages (see Egholm (1992) J. Am. Chem. Soc. 114:1895; Meier et al. (1992) Chem. Int. Ed. Engl. 31: 1008; Egholm et al. (1993) Nature 365:566-568; and Carlsson et al. (1996) Nature 380: 207). Other analog nucleic acids include those with positive backbones (Denpcy et al. (1995) Proc. Natl. Acad. Sci. USA 92: 6097); non-ionic backbones (U.S. Pat. No. 5,386,023 to Sanghvi et al.; U.S. Pat. No. 5,637,684 to Cook et al.; U.S. Pat. No. 5,602,240 to de Mesmaeker et al.; U.S. Pat. No. 5,216,141 to Benner; and U.S. Pat. No. 4,469,863 to Ts'o and Miller; Angew. (1991) Chem. Intl. Ed. English 30: 423; Letsinger et al. (1988) J. Am. Chem. Soc. 110: 4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; de Mesmaeker et al. (1994) Bioorganic & Medicinal Chem. Lett. 4: 395; Jeffs et al. (1994) J. Biomolecular NMR 34:17; Horn et al. (1996) Tetrahedron Lett. 37:743); and non-ribose backbones, including those described in U.S. Pat. No. 5,235,033 to Summerton et al. and U.S. Pat. No. 5,034,506 to Summerton et al., and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp169-176). Several nucleic acid analogs are described in Rawls, Chemical & Engineering News Jun. 2, 1997, page 35. These modifications of the ribose-phosphate backbone can be done to facilitate the addition of additional moieties such as labels, or to increase the stability and half-life of such molecules in physiological environments.

### BRIEF DESCRIPTION OF THE DRAWINGS

FIG. 1 schematically illustrates agent GC-24 contacting a nuclear receptor dimer/heterodimer regulatory site (DHRS). The surface of the human thyroid hormone receptor is in gray, its underlying layers of helices are represented as colored ribbons, and the four side chains of the hydrophobic cluster of amino acids at the RXR (Leu 420- not shown) interface binding site are in green. GC-24 is shown in purple bonds with its surface in gray.

FIG. 2 schematically illustrates the superposition of TRβ/GC-24 and a heterodimer of PPARγ/RXR. GC-24 (red) binds to TR (dark green) at the interface between the dimer partners as indicated by the PPAR (green)/RXR (yellow) heterodimer.

FIG. 3 schematically illustrates the dimerization surface of TR and GC-24II. Polar residues, which are indicated in magenta, and hydrophobic residues, which are indicated in green, that form interactions with a dimer partner are shown. The benzyl of GC-24 builds nonpolar interactions with a cluster of four hydrophobic residues. Superimposed with the benzyl of GC-24 is the side-chain of RXR Leu 420 (green).

FIG. 4 provides another schematic of the environment of the hydrophobic benzyl extension of GC-24 as compared to GC-1, with GC-24 and surrounding side chains shown in beige, and GC-1 shown blue. The residues most changed by GC-24 binding are found at the start of helix 3 and the C-terminus of helix 11.

### DETAILED DESCRIPTION

Normal function of the heart, bone growth, brain development, metabolic regulation, weight maintenance, cholesterol management, normal cell death and regulation all depend on correct function of some or many of the 48 known human nuclear receptors. Nuclear receptors control cell differentiation, development, metabolism and organ physiology by activating or repressing target gene transcription in response to hydrophobic organic molecules, such as steroids, retinoids, vitamin D, thyroid hormone and eicosanoids.

In addition, recent developments in nuclear receptor structure-function illuminate the roles of these receptors in cardiovascular disease, obesity, diabetes, drug metabolism, bone disease, cancer and other diseases. An important goal in the field is the identification of novel small molecules that activate or inhibit the actions of nuclear receptors in specified physiological venues.

Typically, nuclear receptor interacting compounds can be classified agonists, partial agonists-partial antagonists, antagonists, mixed agonist-antagonists or inverse agonists. An agonist can induce changes in receptors that place the receptor in an active conformation, allowing them to influence transcription (either positively or negatively). Most naturally-produced ligands are agonists. On the other hand, classic antagonists bind to the ligand-binding pocket of nuclear receptors and block binding of an agonist. Bound there, they fail to generate the conformational changes in the receptor elicited by the agonist, and either simply block agonist actions or distort the receptor in some other manner. Partial agonists or partial antagonists bind to receptors and yield a response less than that of a full agonist/antagonist at saturating ligand concentrations. Mixed agonists-antagonists act in different ways through the same receptor type depending on context (which cells, which promoter, etc.). The term “inverse agonists” refer to ligands that exert agonist effects that are completely distinct from that of the native ligand. The effects of the compounds vary in different tissues and with respect to the factors that interact with hormone-responsive genes. Thus, the same compound in one tissue or context can act differently in another context,

Synthetic agonists and antagonists have been found for a number of pharmaceutical applications; however, obtaining effective compounds for others have proven to be difficult. For example, it has been difficult to obtain antagonists for nuclear receptors, because of the activation function 1 (AF-1) domain on the amino terminus. For example, binding of an agonist or antagonist to a steroid receptor usually dissociates a heat shock protein (hsp) from the steroid receptor, after which the receptor dimerizes. This event typically activates AF-1. Classical antagonists block activation function 2 (AF-2) domain, and do not directly target AF-1. Thus, when AF-1 is exposed, an AF-2 blocking compound cannot completely inhibit receptor activation (e.g., when AF-1 can function). The invention solves this problem and others by providing compounds that modulate (e.g., activate or repress) nuclear receptors at a site that is outside the ligand binding pocket. This site is termed the nuclear receptor dimer/heterodimer regulatory site (DHRS), which is described herein. Thus, an agent of the invention can block dimerization of a nuclear receptor through the DHRS site and overcome the problem(s) above.

In addition, classical antagonists are analogues of agonists and often have partial agonist activities, as well as problems with cross-reactivity with other nuclear receptors. Because the DHRS is a site that is outside of the ligand binding pocket, the agents that contact the DHRS can be any number of a variety of molecules and do not have to be analogues of agonists.

In addition, the DHRS provides a mechanisms by which to modulate receptors that do not respond (or poorly respond) to classical agonists or antagonists, or that do not have a known ligand. For example, there are situations (e.g., in cancer) where the receptor becomes resistant to classical antagonists. This resistance can be the result, for example, of mutation of the binding site of the nuclear receptor to render an antagonist an agonist, activation of the receptor by second messenger pathways that bypasses a need for ligand binding, and/or mutation of the receptor to a constitutively-active form thereof. For example, in one type of cancer involving thyroid hormone receptors, the unliganded receptors are active and have mutated such that they do not bind ligand. Thus, it would be beneficial to block actions of nuclear receptors for which a ligand is not needed, or for a nuclear receptor for which a ligand has not been found. Thus, this invention provides alternate ways to block actions of either liganded or unliganded receptors.

The agents that interact with the DHRS can also modulate receptors by inferring with cofactor molecules, e.g., coactivators or corepressors, that are bound, or that are binding, to the nuclear receptor. For example, an agent that contacts the DHRS can block the binding of the corepressor, or can remove the corepressor, thereby activating the receptor.

The invention provides methods and for identifying, designing, and/or producing agents (modulators) for nuclear receptors, along with nuclear receptor:modulator complexes and libraries of modulators. In addition, the invention provides novel types of pharmaceuticals to modulate nuclear receptors.

Nuclear Receptors

There are important clinical reasons for modulating nuclear receptor action. For example, there are known conditions in which an excess of a hormone that interacts with a nuclear receptor causes deleterious effects, such as with hyperthyroidism (acting through the thyroid hormone receptor, TR), primary and secondary aldosteronism (acting through the mineralocorticoid receptor, MR), spontaneous Cushing's syndrome (acting through the glucocorticoid receptor, GR), and some cases of hirsutism (acting through the androgen receptor, AR). In addition, hormone-dependent cancers such as those of the breast (estrogen receptor, ER), prostate (AR), thyroid cancer, bone cancer, and ovarian cancer can be treated by modulating nuclear receptor activation, along with other diseases involving nuclear receptors, e.g., hypercholesterolemia, hyperlipidermia, atherosclerosis, obesity, cardiac arrhythmia, hypothyroidism, osteoporosis, hypertension, glaucoma, depression, etc.

A long standing but previously distant goal for pharmaceutical companies has been to modulate protein interactions of nuclear receptors, because the nuclear receptors typically modulate transcription as homodimers or heterodimers. Homodimer function is the norm for the steroid receptors, e.g., AR, GR, MR, ER, and progesterone receptor (PR). TR, vitamin D receptor (VDR), retinoic acid receptor (RAR), peroxisome proliferator activated receptor (PPAR), and many other nuclear receptors partner with a “master control” receptor, such as the retinoid X receptor (RXR), to form heterodimers which inhibit or stimulate transcription. The heterodimer interface has been defined to some extent through X-ray crystallography and mutational analyses. For example, structures are known for PPAR-RXR and RAR-RXR. From these studies, it was found that side chains, predominantly residues of helices 9, 10 and 11, form both polar and nonpolar bonds between the dimer partners. In addition, mutagenesis studies on RXR and TR revealed several residues that are important for TR-RXR dimer formation, and allowed for definition of a dimer/heterodimer interphase.

However, most pharmaceutical companies have abandoned programs in blocking protein function by interfering with protein associations such as the heterodimer TR-RXR for various reasons. For example, the interface between two receptor monomers can be difficult to define except by X-ray crystallography, and also is very large (typically a few thousand Å²). Generally, pharmaceuticals cannot cover more than a hundred or two hundred Å². In addition, the binding energy distributed over the entire interface may be on the order of 10 kcal per mole, a large value to expect competition from a small molecule. Furthermore, the interface is comprised of segmented interleaved surfaces.

This invention solves these problems by providing a surprising and previously unknown specific site, termed the nuclear receptor dimer/heterodimer regulatory site (DHRS), that modulates receptor dimerization/heterodimerization and/or interactions of a nuclear receptor with cofactor molecules, e.g., binding of cofactor molecules (e.g., Barry et al. Journal of Biological Chemistry 2003, in press) and nuclear receptor folding, e.g., appropriate folding of the C-terminal F-domain of the steroid receptors against the ligand binding domain (e.g., Sack et al. PNAS. (2001) 98, 4904-4909). The DHRS is a specific defined site. This site was previously never distinguished as a potential pharmaceutical target. Thus, the DHRS is a critical site that can be exploited to bind appropriately designed agents. Such sites are not easily identified and are known as hot spots. The present invention provides methods for identifying, designing, and/or producing compounds or agents, e.g., modulators, for nuclear receptors along with nuclear receptor modulator complexes and libraries of modulators that bind to this site. Compositions of nuclear receptor modulator complexes, along with libraries of modulators are also provided. Thus, this invention provides a way of modulating protein associations of nuclear receptors.

Receptor Domain Organization

Nuclear hormone receptors are single polypeptide chains that have a similar domain organization. The receptors are organized with an amino terminal A/B domain (sometimes referred to as a variable amino-terminal domain), a highly conserved central DNA binding domain comprising two zinc fingers (DBD) and a hinge region, and a carboxy-terminal ligand binding domain (LBD). Details on the organizational structure of nuclear hormone receptors such as the thyroid receptor are found in Yen (2001), supra. Gene sequences of representative nuclear receptors or their ligand binding domains have been cloned and sequenced, including the human RAR-alpha, human RAR-gamma, human RXR-alpha, human RXR-beta, human PPAR-alpha, human PPAR-beta, human PPAR-gamma, human VDR, human ER (as described in Seielstad et al. (1995) Molecular Endocrinology, 9:647-658), human TR-α, human TR-β, human GR, human PR, human MR, and human AR, as well as mouse and/or rat or other homologues for many of these. The ligand binding domain of each of these nuclear receptors has been identified. This invention identifies a new site on a nuclear receptor involved in dimerization/heterodimerization and/or cofactor molecule interactions, which is not part of the ligand or hormone binding site. The DHRS site can change its structure and recognition propensities when hormone is bound or not through allosteric mechanisms. Because this site is part of the docking site for partner proteins that affect the transcriptional activity of the nuclear receptor, this site is termed a nuclear receptor dimer/heterodimer regulatory site (DHRS).

Nuclear Receptor Dimer/Heterodimer Regulatory Site (DHRS)

The nuclear receptor dimer/heterodimer regulatory site (DHRS) is a hydrophobic cluster of amino acids that is found on the surface of a nuclear receptor. This site is involved in dimerization/heterodimerization of nuclear receptors. It can also be involved in interactions with cofactor molecules of a nuclear receptor. Thus, agents that contact this site can modulate nuclear receptor activation, e.g., by activating or blocking activation.

This site can optionally include neighboring amino acids. In one embodiment, the DHRS is composed of other regions. For example, a DHRS can include a first region, which is the hydrophobic cluster or indent, e.g., as delineated in Table 1, and a second region, which can include polar and/or non-polar amino acids proximal to the hydrophobic cluster. An optional third region includes a solvent-accessible (i.e., “solvent-based”) region. This provides an environment that permits flexibility in compound design, e.g., to allow design criteria that can be needed for bioavailability or solubility. For example, GC-24 bound to thyroid hormone beta receptor (TR) demonstrates the utilization of all three regions described above for TR. The regions are as follows:

Region 1: Val376, Leu400, Leu422, Val425;

Region 2: Ser381, Asp382, Glu393, Glu396, Arg429; and/or

Region 3: solvent molecules (for example, water molecules that interact with the proximal benzyl and carboxylate of compound GC-24).

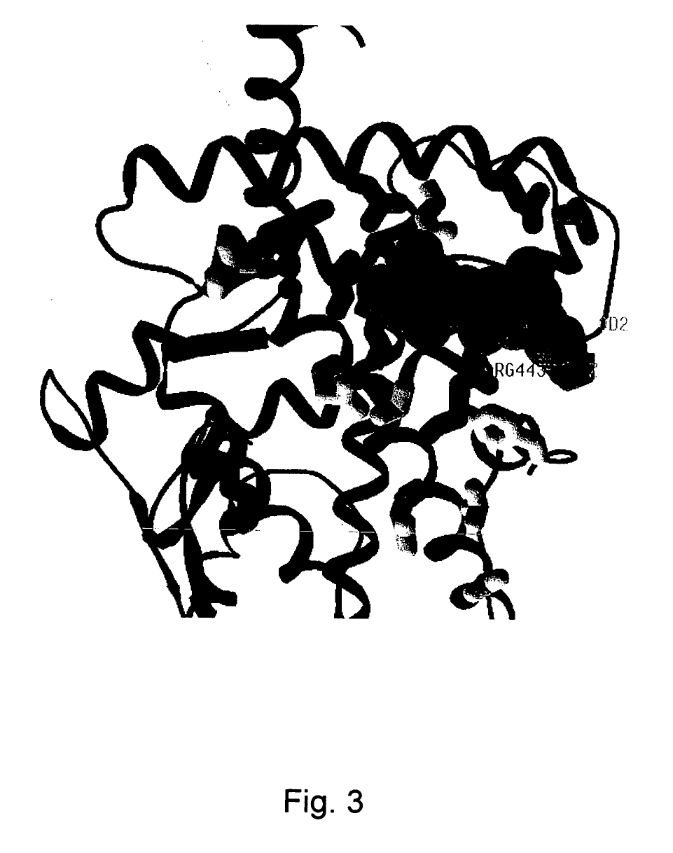

For example in FIG. 3, the DHRS site lies near the northeast on the receptor dimerization interface, in the standard orientation shown in the figure. In this example, the DHRS comprises those amino acids on the surface of a receptor which form dimers with the nuclear receptor RXR. Certain receptors such as estrogen receptor mimic this interaction but are of the form NR:NR rather than RXR:NR. In another example that includes PPARγ, the amino acids that contact RXR (e.g., being situated less than 4.5 A away in the crystal structure of the dimer) can include 24 residues: e.g., K373, G395, D396, P398, V403, E407, Q410, L414, E418, S429, Q430, F432, A433, K434, 1436, Q437, M439, T440, D441, R443, Q444, T447, Q451, and Y477.

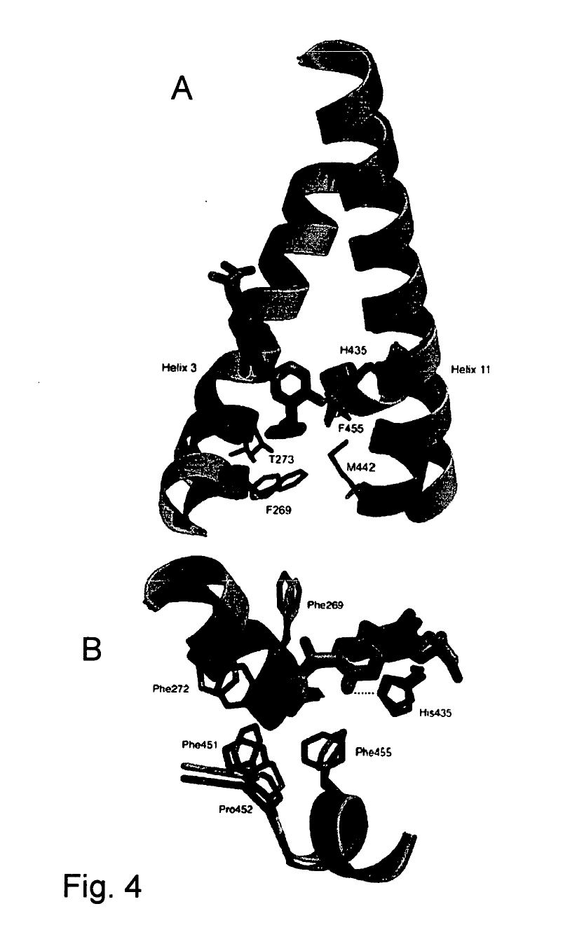

FIG. 4 provides a schematic depiction of the LBS of TR, showing the environment of the hydrophobic benzyl extension of GC-24. GC-24 and the surrounding side chains are shown in beige, while GC-1 (3,5-dimethyl-4-(4′-hydroxy-3′-isopropyl)benzyl phenoxy acetic acid) is shown blue. The residues most altered by GC-24 binding are positioned at the start of helix 3 and the C-terminus of helix 11. Phe 451, Pr 452, Phe 455 and, to a lesser extent, Ile276 (not shown) enhance the hydrophobic cluster linking helix 11 and helix 12 to the receptor core (only in GC-24). The benzyl extension is depicted as forming close packing interactions with six hydrophobic side chains.

Other examples of DHRS sites can be found in Table 1. This table includes, but is not limited to, the amino acids forming the DHRS for the nuclear receptors listed. In one example, the DHRS forms a hydrophobic cleft and binds to the compound GC-24. In addition, the region(s) can be determined by, e.g., the three dimensional structure of a nuclear receptor. For example, a DHRS, when mapped to the three dimensional structures for other nuclear receptors, e.g., androgen receptor, binds to the C-terminal strand following helix 12 and binds to a critical Phe side chain. The region can also be determined by where an agent, which is known to bind to a DHRS, binds.

TABLE 1
AMINO ACIDS FORMING REGION I OF THE DHRS FOR
SEVERAL NUCLEAR RECEPTORS

| Receptor | Position | Position | Position | Position |
|---|---|---|---|---|
| TRβ | Val 376 | Leu 400 | Leu 422 | Val 425 |
| TRα | Val 322 | Leu 346 | Leu 368 | Val 371 |
| PPARα | Ala 381 | Val 405 | Leu 427 | Met 430 |
| PPARγ | Val 390 | Leu 414 | Leu 436 | Met 439 |
| RARα | Ile 332 | Leu 356 | Leu 378 | Ile 381 |
| PXR | Ile 346 | Ala 370 | Met 394 | Leu 397 |
| Vit. D | Ile 336 | Ser 360 | Ile 384 | Leu 387 |
| AR | Leu 810 | Ile 835 | Thr 860 | Leu 863 |
| ER | Ile 451 | Thr 483 | Leu 508 | Leu 511 |
| PR | Leu 824 | Ile 849 | Thr 874 | Leu 877 |

As described herein, the DHRS of a nuclear receptor can be expressed, crystallized, its three dimensional structure determined with an agent bound (either using crystal data from the same receptor or a different receptor or a combination thereof), and computational methods used to design agents that contact the DHRS, including modulators, as described herein. Known crystallization or co-crystallization methods can be applied for this purpose, as can the specific methods described herein.

### EXAMPLE

### DHRS for β Thyroid Receptor

In this example, the properties of a TR interacting compound (GC-24, 3, 5-dimethyl-4-(4′-hydroxy-3′-benzyl)benzyl-phenoxy acetic acid) were determined. We solved the crystal structure of GC-24 in complex with TRβ, the atomic coordinates for which are provided in Appendix I. The X-ray crystal structure of GC-24 complexed with the TR LBD revealed two molecules of GC-24 bound to each LBD. The first molecule is bound in the ligand-binding pocket of the LBD (see U.S. Ser. No. 10/317,034 entitled “A method for creating specific, high affinity nuclear receptor pharmaceuticals” by Baxter et al., filed Dec. 10, 2002 and PCT publication filed December 2003 Attorney Docket number 407J-000520PC). The second molecule bound to the nuclear receptor surface (the DHRS of the invention), which is the area indicated in bold print in Appendix I. This second site contains GC-24 bound in a surface pocket that is part of the dimer/heterodimer interface. When GC-24 binds to this site, the TR is prevented from complexing with another nuclear receptor, e.g., RXR. Thus, the new site expands the ability to modulate a nuclear receptor by acting at this site.

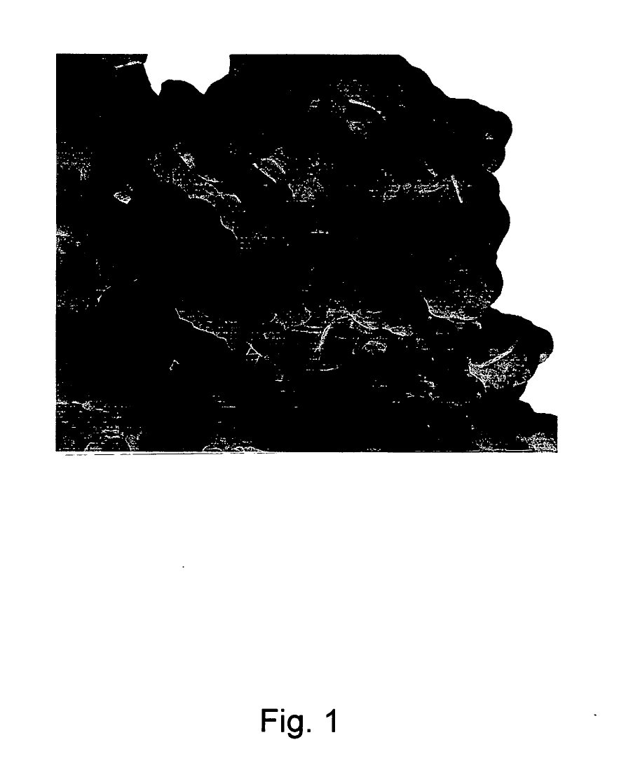

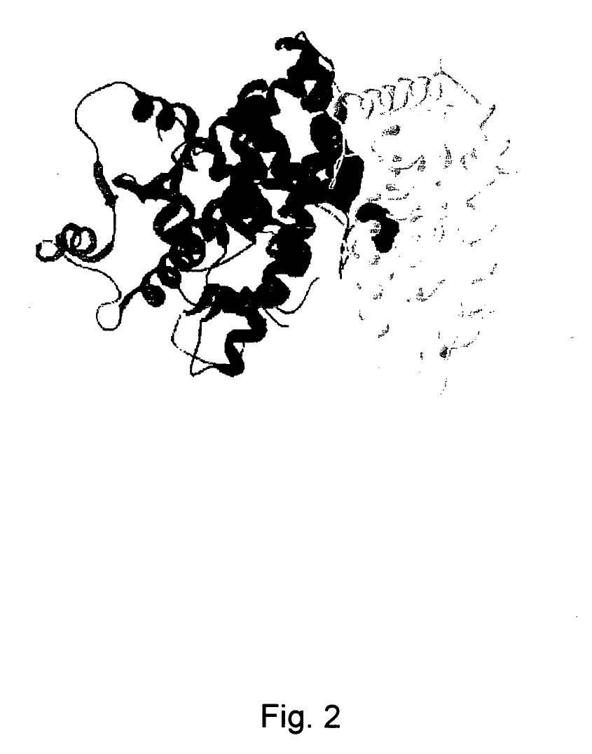

FIGS. 1-3 show binding of GC-24 to TR nuclear receptor dimer/heterodimer regulatory site. FIG. 1 shows a close up surface of the nuclear receptor dimer/heterodimer regulatory site of the thyroid hormone receptor with GC-24 interacting at this site. FIG. 2 shows the hormone analogue GC-24 at the interface of an RXR-Heterodimer. FIG. 3 shows GC-24 at a nuclear receptor dimer/heterodimer regulatory site.

In the TR, a hydrophobic cluster of amino acid residues Val 376, Leu400, Leu422 and Val425 defines the DHRS (Table 1) as described above. The two structures of heterodimers show this site filled with Leu 420 from RXR. The TR-LBD/GC24 structure was superimposed with heterodimeric structures of PPAR-RXR and RAR-RXR. Published analysis of TR-RXR heterodimer stability showed that this Leu is required for heterodimer formation, but did not explain why or how its absence may function to block dimer formation, since the authors inserted an Arg at this position.

In our structure, GC-24 mimics the Leu 420 of RXR. The presence of the GC-24 blocks formation of the heterodimer. Pharmaceuticals that interact with this site can modulate dimer/heterodimer formation, which will activate or inhibit receptor activation, thereby modulating gene transcription.

Ligand Binding Domain

The ligand binding domain (LBD), of which the DHRS is a part, is the second most highly conserved domain in these receptors. The LBD is comprised of a stack of three helical layers. While the integrity of several different LBD sub-domains is important for ligand binding, truncated molecules containing only the LBD retain normal ligand-binding activity. This domain also participates in other functions, including dimerization (e.g., through the DHRS), nuclear translocation and transcriptional activation. This domain binds the ligand and undergoes ligand-induced conformational changes. See, e.g., U.S. Pat. No. 6,236,946 to Scanlan et al. entitled “Nuclear Receptor Ligands and Ligand Binding Domains” issued May 22, 2001; and, U.S. Pat. No. 6,266,622 to Scanlan et al., entitled “Nuclear Receptor Ligand Binding Domains” issued Jul. 24, 2001.

The LBD is necessary for hormone binding and also plays an important role in basal repression by unliganded receptor, as well as dimerization, and transactivation. The crystal structure of liganded thyroid receptor provides precise information as to ligand binding and function. See, Yen (2001), supra; Bourguet et al. (1995) “Crystal structure of the ligand binding domain of the human nuclear receptor RXR-alpha” Nature 375:377-382; Renaud et al. (1995) “Crystal structure of the RAR-gamma ligand binding domain bound to all-trans retinoic acid”; Nature 378:681-689; Wagner et al. (1995) “A structural role for hormone in the thyroid hormone receptor” Nature 378:690-697; Brzozowski et al. (1994) “Molecular basis of antagonism in the oestrogen receptor” Nature 389:753-758; Darimont et al. (1998) “Structure and specificity of nuclear receptor-coactivator interactions.” Genes Dev 12:3343-3356; Feng et al. (1998) “Hormone dependent coactivator binding to a hydrophobic cleft on nuclear receptors” Science 280:1747-1749; U.S. Pat. No. 6,266,622 “Nuclear receptor ligands and ligand binding domains” by Scanlan et al.; and, Marimuthu et al. (2002) “Thyroid hormone receptor surfaces and conformations required to bind nuclear receptor corepressor (N-CoR)” Mol Endocrinol 16:271-286.

In the unliganded state, nuclear receptors are either bound to heat shock proteins (hsp) in a complex in which they are largely inactive, or are bound to DNA or other proteins involved in transcription control (e.g., usually corepressor proteins that either repress or stimulate transcription). Binding of the hormone releases the heat shock proteins or corepressor and results in the folding of helix 12, the terminal helix of the LBD, into the body of the receptor, where it forms part of the coactivator-binding surface. Depending on whether or not the hormone is bound, helix 12 acts like a switch that turns genes on or off.

In the LBD, ligand is buried within a mostly hydrophobic pocket formed by discontinuous stretches spanning the LBD. The most carboxy-terminal region (helix 12) contributes its hydrophobic surface as part of the ligand binding pocket. The hydrophobic residues face inwards, whereas conserved glutamate residues of the helix face outwards. The pocket is bounded by hydrophobic surfaces from helixes 3, 4, and 5. The crystal structure of the unliganded RXR receptor shows that helix 12 projects into the solvent, closing in “mouse trap” fashion on the ligand once bound. Helix 12 of raloxifene-bound ER LBD is in a different position, lying in a groove between helices 3 and 5. Thus, the relative positions of helix 12 and the boundary helixes determine whether coactivators can interact with a given receptor.

a) Activation Subdomains

Most members of the nuclear receptor superfamily, including orphan receptors, possess at least two transcription activation subdomains, one of which is constitutive and resides in the amino terminal domain (AF-1), and the other of which (AF-2, also referred to as TAU 4) resides in the ligand-binding domain and whose activity is regulated by binding of an agonist ligand. Although the activity of AF-1 is not directly activated by ligand binding, it can be activated indirectly. For example, unliganded steroid hormone receptors are bound by heat shock proteins and rendered largely inactive. Binding of an agonist or in some cases antagonist ligand can cause dissociation of the heat shock protein with subsequent binding of the receptor to proteins or DNA where the AF-1 function can be active. Unlike classical agonists or antagonists, this invention provides methods to modulate AF-1. For example, agents that contact the DHRS site can modulate dissociation of the heat shock protein, thereby inhibiting activation of AF-1 or by activating AF-1.

The function of AF-2 requires an activation domain (also called transactivation domain) that is highly conserved among the receptor superfamily. Most LBDs contain this activation domain. Some mutations in this domain abolish AF-2 function, but leave ligand binding and other functions unaffected. Ligand binding allows the activation domain to serve as an interaction site for essential coactivator proteins that function to stimulate (or in some cases, inhibit) transcription. Based upon the structure of TRs, the activation domain is proposed to adopt an amphipathic helical structure. β-sheet or mixed secondary structures, can be present as activation domains in less related nuclear receptors.

Within the activation domain, the highly conserved motif ΦΦXEΦΦ, where Φ represents a hydrophobic residue, mediates interactions between the receptors and transcriptional coactivators. Several proteins have been identified which bind the TR in a hormone-dependent fashion. One of these, Trip1, is related to a putative yeast coactivator Sugl, and also interacts with both the C-terminal activation domain and a subset of the basal transcriptional machinery, suggesting a role in transactivation by the TR. Other proteins, such as RIP140, SRC1, (Onate et. al. (1995) Science 270:1354-1357), TF-1 (Ledouarim et. al. (1995) EMBO J. 14:2020-2033), GRIP-1 (Heery et al. (1997) Nature 387:733-736) and TRAP220 (Fondell et al. (1996) Proc. Natl. Acad. Sci. USA 93:8329-8333) also interact with other nuclear receptors in a ligand dependent manner through the C-terminal domain. Binding of these proteins can be modulated using the agents (modulators) of the invention described herein.

The role of coactivators and corepressors in steroid/thyroid hormone receptor systems is well known. See, for example, Shibata et al. (1997) Recent Progress in Hormone Res. 52:141-164 for a review. Steroid receptor coactivator-one (SRC-1) appears to be a general coactivator for all AF-2 domain containing receptors tested. SRC-1 enhances transactivation of hormone-dependent target genes. Upon binding of agonist, the receptor changes its conformation and enables recruitment of coactivators such as SRC-1, which allows the receptor to modify chromatin and interact with the basal transcriptional machinery more efficiently and to activate or repress transcription. In contrast, binding of antagonists induces either a different conformational change or no change in the receptor. Although most antagonist-bound receptors can dimerize and bind to their cognate DNA elements, they typically fail to dislodge the associated corepressors, which results in a nonproductive interaction with the basal transcriptional machinery.

Other putative coactivators have been reported, including the SRC-1 related protein TIF-2, GRIP-1, pCIP/ACTR/AIB1, and other putative unrelated coactivators such as TRAP220, ARA-70, Trip 1, PGC-1, and TIF-1. In addition, another coactivator CREB-binding protein (CBP) has been shown to enhance receptor-dependent target gene transcription. CBP and SRC-1 interact and synergistically enhance transcriptional activation by the ER and PR. A ternary complex of CBP, SRC-1, and liganded receptors may form to increase the rate of hormone-responsive gene transcription. Corepressors for TR and RAR, such as SMRT and N-CoR, have been identified that also contribute to the silencing function of unliganded TR. The unliganded TR and RAR have been shown to inhibit basal promoter activity; silencing of target gene transcription by unliganded receptors is mediated by these corepressors. It should be noted that coactivators such as GRIP1 can mediate negative effects on agonist-bound nuclear receptors on negatively regulated genes, and corepressors can mediate positive effects of unliganded receptors on negatively regulated genes.

The collective data suggests that upon binding of agonist, the receptor changes its conformation in the ligand-binding domain thereby enabling recruitment of coactivators, which allows the receptor to interact with the basal transcriptional machinery more efficiently and to activate transcription. In contrast, binding of antagonists induces a different (or no) conformation change in the receptor.

Similarly, TR and RAR associate with corepressors in the absence of ligand, thereby resulting in a negative interaction with the transcriptional machinery that silences target gene expression. In the case of mixed agonist/antagonists, such as 4-hydroxytamoxifen, activation of gene transcription may depend on the relative ratio of coactivators and corepressors in the cell, or cell-specific factors that determine the relative agonistic or antagonistic potential of different compounds. These coactivators and corepressors act as an accelerator and/or a brake that modulates transcriptional regulation of hormone-responsive target gene expression.

Binding of these (and other) coactivators and corepressors can be modulated/regulated using the agents (modulators) of the invention described herein. For example, the modulators of the invention can modulate the level of dimer/heterodimer formation, or can modulate the interaction (association or disassociation) of a nuclear receptor and a cofactor molecule. This can in turn modulate transcription of a gene, e.g., a nuclear receptor responsive gene.

Agents (modulators) of the invention can also be used to distinguish the mechanisms of action of a nuclear receptor. Nuclear receptors appear to work by at least two mechanisms. In the first mechanism, the nuclear receptors bind DNA and regulate the transcription of a first set of genes. In the second mechanism, the nuclear receptors interact with other proteins (e.g., AP-1, NF-KB, etc.) and regulate the transcription of another set of genes. Under the first mechanism, nuclear receptors typically operate as dimers, while in the second mechanism, the nuclear receptor can act as a monomer. Thus, agents of the invention that modulate dimer/heterodimer formation can be used to distinguish the two mechanisms.

b) Carboxy-Terminal Activation Subdomain

The carboxy-terminal activation subdomain is in close three dimensional proximity in the LBD to the ligand, so as to allow for ligands bound to the LBD to coordinate (or interact) with amino acid(s) in the activation subdomain.

DNA Binding Domain

The DNA binding domain (DBD) is the most conserved structure in the nuclear receptor superfamily. It usually contains about 70 amino acids that fold into two zinc finger motifs, wherein a zinc ion coordinates four cysteines. DBDs contain two perpendicularly oriented α-helixes that extend from the base of the first and second zinc fingers. The two zinc fingers function in concert with non-zinc finger residues to direct nuclear receptors to specific target sites on DNA, and to align receptor homodimer or heterodimer interfaces. Various amino acids in the DBD influence spacing between the two half-sites (usually comprised of six nucleotides) for receptor dimer binding. For example, GR subfamily and ER homodimers bind to half-sites spaced by three nucleotides and oriented as palindromes. The optimal spacings facilitate cooperative interactions between the DBDs, and D box residues are part of the dimerization interface.

The LBD can influence the DNA binding characteristics of the DBD, and this influence can also be regulated by ligand binding. For example, TR ligand binding influences the degree to which a TR binds to DNA as a monomer or dimer. Such dimerization also depends on the spacing and orientation of the DNA half sites. This can also be regulated by agents of the invention.

The nuclear receptor superfamily has been subdivided into two subfamilies on the basis of DBD structures, interactions with heat shock proteins (hsp), and ability to form heterodimers: 1) the GR subfamily (including GR, AR, MR and PR) and 2) the TR subfamily (including TR, VDR, RAR, RXR, and most orphan receptors). GR subgroup members are tightly bound by hsp in the absence of ligand, dimerize following ligand binding and dissociation of hsp, and show homology in the DNA half sites to which they bind. These half sites also tend to be arranged as palindromes. TR subgroup members tend to be bound to DNA or other chromatin molecules when unliganded, can bind to DNA as monomers and dimers, but tend to form heterodimers, bind DNA elements with a variety of orientations and spacings of the half sites, and also show homology with respect to the nucleotide sequences of the half sites. However, ER does not belong to either subfamily using this approach to classification, since it resembles the GR subfamily in hsp interactions, and the TR subfamily in nuclear localization and DNA-binding properties.

Amino Terminal Domain

The amino terminal domain is the least conserved of the three domains and varies markedly in size among nuclear receptor superfamily members. For example, this domain contains 24 amino acids in the VDR and 603 amino acids in the MR. Th amino terminal domain is involved in transcriptional activation; in some cases, its uniqueness dictates selective receptor-DNA binding and activation of target genes by specific receptor isoforms. The amino terminal domain can display either synergistic or antagonistic interactions with the domains of the LBD. For example, studies with mutated and/or deletion-containing receptors show positive cooperativity of the amino and carboxy terminal domains. In some cases, deletion of either of these domains will abolish the receptor's transcriptional activation functions.

Types of Nuclear Receptors

The invention can be used to identify, design, produce, etc., modulators for a variety of nuclear receptors, such as receptors for glucocorticoids (GRs), androgens (ARs), mineralocorticoids (MRs), progestins (PRs), estrogens (ERs), thyroid hormones (TRs), vitamin D (VDRs), retinoid (RARs and RXRs), and peroxisome proliferator activated receptors (PPARs)). For example, a nuclear receptor of the present invention includes, but is not limited to, a thyroid hormone receptor, a β thyroid hormone receptor, an alpha thyroid hormone receptor, a glucocorticoid receptor, an estrogen receptor, an androgen receptor, a mineralocorticoid receptor, a progestin receptor, a vitamin D receptor, a retinoid receptor, a retinoid X receptor, a peroxisomal proliferator activated receptor, an estrogen-receptor related receptor, a short heterodimer partner, a constitutive androstane receptor, a liver X receptor, a pregnane X receptor, a HNF-4 receptor, a farnesoid X receptor (FXR) and an orphan receptor. Nuclear receptors can include nuclear receptors expressed by human and non-human species including vertebrates and invertebrates. A database of nuclear receptors is available on the World Wide Web at receptors.ucsf.edu/NR/multali/multali.html.

The invention can also be applied to “orphan receptors,” that are structurally homologous (in terms of modular domains and primary structure) to classic nuclear receptors, such as steroid and thyroid receptors, e.g., a liver orphan receptor (LXR), a farnesoid X receptor (FXR), etc. The amino acid homologies of orphan receptors with other nuclear receptors range from very low (<15%) to in the range of about 35% when compared to rat RAR-α and human TR-β receptors, for example. In addition, as revealed by the X-ray crystallographic structure of the TR and structural analysis, the overall folding of liganded superfamily members is similar. See, U.S. Pat. No. 6,236,946 to Scanlan et al. entitled “Nuclear Receptor Ligands and Ligand Binding Domains” issued May 22, 2001; and, U.S. Pat. No. 6,266,622 to Scanlan et al., entitled “Nuclear Receptor Ligand Binding Domains” issued Jul. 24, 2001. One skilled in the art can apply the invention to the identification, design, production, etc, of one or more modulators that contact the DHRS from a selected orphan receptor, as these receptors' overall structural modular motif is similar to other nuclear receptors.

Isoforms

The invention is also applicable to generating modulators that display differential activity on nuclear receptor isoforms. That is, an agent of the invention can increase specificity as well as affinity, including specificity to distinguish between/among different forms of a DHRS of a given receptor. The term “isoform” refers to closely-related receptors, which can be products of distinct genes or products of differential splicing from the same gene. In general, isoforms encode receptors that would be assigned to the same class, e.g., isoforms α1, α2, β1, and β2 for TR, isoforms α, β, γ for PPAR, isoforms αand β for ER in humans, and isoforms α and β and gamma ER in fish. The isoforms often bind the same ligand, but can also differ in their affinity of binding to particular ligands. In one embodiment of the invention, it is desirable to design agents (modulators) that bind to and act selectively through, e.g., one isoform's DHRS.

As described herein, modulators of the invention can be generated that distinguish between different receptors or different isoforms of a given receptor, thereby allowing the generation of, e.g., tissue-specific or function-specific modulators (or both). For instance, GR subfamily members usually comprise one receptor encoded by a single gene, although there are certain exceptions. For example, there are two PR isoforms, A and B, translated from the same mRNA by alternate initiation from different AUG codons. There are two GR forms, one of which does not bind ligand. In another example, the TR subfamily has several receptors that are encoded by at least two genes (TR: α, β) or three genes (RAR, RXR, and PPAR: α, β, γ) and/or that arise due to alternate RNA splicing. See, Yen (2001), supra, for a review of TR receptor isoforms.

In one aspect, the invention includes methods for identifying, designing, producing, etc. a compound having modulator activity on a nuclear receptor, e.g., in an isoform-specific manner, e.g., on a thyroid hormone receptor (TR). A “TR isoform” includes TR proteins encoded by subtype and variant TR genes. This includes TR-α and TR-β isoforms encoded by different genes (e.g., TRα and TRβ) and variants of the same genes (e.g., TRβ1 and TRβ2).

Compounds of the Invention

An agent or modulator of the present invention contacts the nuclear receptor dimer/heterodimer regulatory site (DHRS) of the nuclear receptor. The agent will have a region that fits within the DHRS with some flexibility, and optionally interacts with the residues of the site. Typically, the agent modulates, e.g., inhibits, dimer/heterodimer formation of nuclear receptors, which can lead to modulation of the nuclear receptor. The agent can also modulate nuclear receptor and cofactor molecule interactions. For example, the agent can either inactivate the nuclear receptor or activate the nuclear receptor, e.g., by interfering with cofactor molecules, such as a corepressor or coactivator. In certain embodiments, an agent of the invention modulates nuclear receptor activation by modulating nuclear receptor conformation that, e.g., stabilizing or destabilizing binding of the ligand with the nuclear receptor. For example, an agent of the invention can modulate the off-on rate (e.g., increase off rate, increase on rate, decrease off rate, or decrease on rate) of the ligand to the nuclear receptor as compared to a control. These properties, along with others, can be measured by standard binding procedures or assays, e.g., by performing protein-protein interaction assays, by calculating or testing binding energies computationally, or using thermodynamic or kinetic methods as known in the art.

Nuclear Receptor Complexes

In a further aspect, the present invention provides nuclear receptor:modulator complexes. The receptor:modulator complex includes a nuclear receptor bound to an agent, where the agent preferentially binds a nuclear receptor dimer/heterodimer regulatory site of a nuclear receptor. For example, an exemplary agent for use in the nuclear receptor:modulator complexes of the present invention includes a molecule GC-24. Alternatively, the agent is other than GC-24.

Complexes of the invention can be formed or used in vitro or in vivo, or a combination of both. For example, the complex can be in a container, or alternatively, a cell, or an organism, e.g., a mammal, such as a human. Optionally, the nuclear receptor is inactivated in the nuclear receptor:modulator complex. Alternatively, the nuclear receptor is activated in the nuclear receptor:modulator complex.

Libraries of modulators for a nuclear receptor are also included in the invention. See, Libraries of Modulators section below.

Identifying, Designing and Producing Modulators

Method of identifying, designing and/or producing agents that contact the nuclear receptor dimer/heterodimer regulatory site are also provided. In one embodiment, the modulator is GC-24. Alternatively, the modulator is a modulator other than GC-24.

Screening and/or Identifying Agents that modulate Dimer/Heterodimer Formation or Cofactor Molecule Interactions

Methods for screening for a test agent that modulates dimer/heterodimer formation or cofactor molecule interactions of nuclear receptors are included in the present invention. For example, the methods include contacting at least one nuclear receptor dimer/heterodimer regulatory site (DHRS) of at least one nuclear receptor with a test agent; and, detecting a change in level of dimer/heterodimer formation and/or detecting a change in cofactor interactions of the at least one nuclear receptor that is mediated by the test agent, e.g., as compared to a control. In one embodiment, the at least one nuclear receptor comprises at least two nuclear receptors (e.g., where one of the at least two nuclear receptors is a retinoid X receptor (RXR)). Any of above steps can be performed in vitro, or in vivo, or in any combination thereof. For example, steps of the method (or agents of the present invention produced by binding of the agent to the DHRS) can be in a cell-free in vitro system (e.g., a transcription/translation system), or in a cell, or in a mammal. An agent and/or a library that includes a plurality of different agents produced by this method are also included in the invention.

In further aspects, the change in the level of dimer/heterodimer formation of the at least one nuclear receptor can be compared to the level of dimer/heterodimer formation in a control, e.g., where the difference in the level of dimer/heterodimer formation in the contacted DHRS and the level in the control indicates that the agent alters dimer/heterodimer formation of the at least one nuclear receptor. In certain embodiments, the control is exposed to a lower concentration of test agent, or no test agent.

In further embodiments, the change in the interactions of cofactor molecules with the at least one nuclear receptor can be compared to the interactions in a control, e.g., where the difference in the interactions in the contacted DHRS and the interactions in the control indicates that the agent alters or modulates the interactions of the at least one nuclear receptor with cofactor molecules. In certain embodiments, the control is exposed to a lower concentration of, or no, test agent.

Other methods of identifying one or more agents (modulators) for at least one nuclear receptor (and the modulators identified by the methods) are provided. In the methods of the present invention, a plurality of putative modulators are provided, the plurality of modulators are contacted to at least one nuclear receptor dimer/heterodimer regulatory site (DHRS) of a nuclear receptor, where at least one of the putative modulators binds the DHRS, and, the putative modulators are tested for modulator activity on the nuclear receptor, thereby identifying the one or more modulators of the nuclear receptor. The plurality of putative modulators can range in population from tens to thousands (e.g., the plurality includes, but is not limited to, sets having about 5, 10, 50, 100, 500, 1000 or more members).

The agents that fit into the DHRS of this invention need not look like generic hormones, e.g., so they will not adversely cross-react. In one embodiment, the same class of agents can be used to make drugs for all nuclear receptors that utilize the DHRS. In another embodiment, the agent can be specific for a certain nuclear receptor, a particular isoform of a nuclear receptor, etc.

Indeed, virtually any test agent can be screened as an agent that modulates nuclear receptor dimerization/heterodimerization and/or cofactor molecule interactions, or as a putative modulator according to the methods of this invention. Such test agents include, but are not limited to, small organic molecules, nucleic acids, proteins (e.g., polypeptide, antibody, or fragment thereof), peptides, peptide analogs, sugars, polysaccharides, glycoproteins, lipids, and the like. The term “small organic molecules” typically refers to molecules of a size comparable to those organic molecules generally used as pharmaceuticals, and as such excludes biological macromolecules (e.g., proteins, nucleic acids, etc.). In certain embodiments, the test agent is a peptide, e.g., less than 15 amino acids, less than 10 amino acids, less than 8 amino acids, etc. In certain embodiments, the peptide is unrestrained, while in other embodiments, the peptide can be cyclized or constrained. The peptide can be composed of natural, synthetic or a combination of natural and synthetic amino acids. In certain embodiments, the test agent is an agent other than antibody, a protein, or a nucleic acid. The test agent can be contacted directly to the at least one DHRS, or contacted to a cell containing the at least one DHRS, or contacted to an animal comprising a cell containing the at least one DHRS.

A number of assays can be used to screen for agents (or test agents) that contact the DHRS, e.g., assays directed toward nucleic acid expression, protein-protein interactions, etc. Many assays are described in a section herein entitled “Assays for Modulator Activity.” In certain embodiments, the identified DHRS is utilized to screen or identify agents of the invention, including proteins or polypeptides that modify the activity of the nuclear receptors. Such modification can occur by covalent modification, such as by phosphorylation, acetylation, etc., or by protein-protein (homo or heteropolymer) interactions. See also the section entitled Modification. Any methodology suitable for detecting protein-protein interactions can be employed in the methods of the present invention, including, but not limited to, co-immunoprecipitation, cross-linking and co-purification through gradients or chromatographic columns, gel-shift assays, western blots, far western blots, fusion tag assays, capture assays, e.g., using a nonnatural amino acid, two-hybrid (e.g., yeast, mammalian, etc.) systems, etc.

Co-immunoprecipitation techniques utilizing an antibody that recognizes, e.g., the DHRS, can be used to immunoprecitate the DHRS along with any proteins (which are typically labeled) that interact or contact the DHRS. These complexes can be isolated with, e.g., Protein A-, Protein G-, or antibody-bound beads or resin. These proteins can be analyzed by, e.g., SDS-PAGE, western blots, etc. Far western blots can be used to identify proteins that interact with the DHRS by interacting labeled proteins or polypeptides with a western blot containing a membrane-bound renatured protein or polypeptide comprising DHRS.

In fusion tag assays, gene products can be expressed in vivo as fusion protein. See, e.g., Chinnaiyan, A. M., et al., (1995), Cell, 81:505. For example, a DHRS fusion protein can be generated. Proteins or polypeptides that interact with the fusion protein, e.g., the DHRS fusion protein, are screened. Typically, proteins or polypeptides being screened to identify those that interact with the protein component of the fusion protein are typically labeled, e.g., radiolabeled, nonradiolabeled (e.g., biotinylated). The fusion protein is fused or tagged to another sequence, e.g., a protein or polypeptide sequence comprising the DHRS. Tags include, but are not limited to, glutathione-S-transferase (GST)-tagged, His-tagged (e.g., typically using about 6 or more histidines), epitope-tagged, etc. fusion polypeptides. These fusion proteins can be bound to an affinity matrix, beads, cellulose matrix, cellulose beads, agarose matrix, agarose beads, Sepharose-beads, magnetic beads, glutathione-bound matrix or beads, nickel (e.g., NTA, IDA, etc.) matrix or resin, etc. The labeled proteins or polypeptides that interact with the fusion protein can be isolated by mixing or interacting the labeled proteins or polypeptides with fusion proteins bound to the affinity matrix, beads, etc. Those labeled proteins or polypeptides that do not interact with the fusion protein are washed away, while the bound labeled protein or polypeptide can be eluted (e.g., by excess glutathione) and, optionally further analyzed. Agents that interact with the DHRS can be identified using a fusion-tag assay.

Capture assays can be used to screen for agents that contact or interact with the DHRS. In a capture assay, a protein or polypeptide comprising DHRS is made, which incorporates an unnatural amino acid, e.g., a biotinylated lysine residue. The protein containing the biotinylated lysine residue can then bind an avidin- or streptavidin-linked bead, e.g., streptavidin-linked agarose, streptavidin-linked magnetic beads, etc. Proteins or polypeptides that interact or contact the DHRS that is bound to the beads via the biotinylated lysine residue and the (strept)avidin-linked bead can be captured by mixing test agents with the beads. The beads are washed and the bound captured agents that contact or that interact with the DHRS can be eluted and identified.

The two-hybrid system detects protein interactions in vivo and is described in, e.g., Chien, et al. (1991) Proc. Natl. Acad. Sci. USA, 88, 9578-9582 and is commercially available from Clontech (Palo Alto, Calif.). For example, plasmids are constructed that encode two hybrid proteins: one consists of the DNA-binding domain of a transcription activator protein fused to, e.g., the DHRS, and the other consists of the activation domain of a transcription activator protein fused to an unknown protein that is encoded by a cDNA that has been recombined into the plasmid as part of a cDNA library. The DNA-binding domain fusion plasmid and the cDNA library are transformed into a strain of the yeast Saccharomyces cerevisiae or a mammalian cell that contains a reporter gene (e.g., lacZ) whose regulatory region contains the transcription activator's binding site. Either hybrid protein alone cannot activate transcription of the reporter gene. Interaction of the two hybrid proteins reconstitutes the functional activator protein and results in expression of the reporter gene, which is detected by an assay for the reporter gene product. The library plasmids responsible for reporter gene expression are isolated and sequenced to identify the proteins encoded by the library plasmids. After identifying proteins that interact with the DHRS, assays for compounds that interfere with the DHRS protein-protein interactions can be performed.

In addition to the references noted supra, a variety of protein methods are well known in the art, including, e.g., those set forth in, e.g., Bollag et al. (1996) Protein Methods, 2nd Edition Wiley-Liss, NY; Walker (2002) The Protein Protocols Handbook, 2^{nd} Edition Humana Press, NJ, Harris; Walker (2002) Protein Protocols on CD-ROM Humana Press, NJ; and, Golemis, (2001) Protein-Protein Interactions: A Molecular Cloning Manual, Cold Spring Harbor Laboratory, NY, and the references cited therein.

Prescreening Test Agents

Methods of prescreening agents that modulate dimer/heterodimer formation and/or cofactor molecule interactions of nuclear receptors are also included in the present invention. The methods include the steps of a) contacting a nuclear receptor dimer/heterodimer regulatory site (DHRS) with a test agent; and b) detecting specific binding of the test agent to said DHRS. In some embodiments, the specific binding indicates that the test agent is a candidate modulator of dimer/heterodimer formation.

In one embodiment, such pre-screening is accomplished with simple binding assays. Means of assaying for specific binding or for determining the binding affinity of a particular agent for a protein are well known to those of skill in the art. In some preferred binding assays, the nuclear receptor with a DHRS (or a polypeptide that includes the DHRS) is immobilized and exposed to a test agent (which can optionally be labeled). Alternatively, the test agent(s) are immobilized and exposed to the DHRS-containing protein or polypeptide. The immobilized moiety is then washed to remove any unbound/non-specifically bound material, and the bound test agent or bound nuclear receptor is examined (e.g. by detection using an assay herein, by detection of a label attached to the bound molecule, or other techniques known to one of skill in the art). Typically, an amount of immobilized label is proportional to the degree of binding between the DHRS and the test agent.

Designing Agents

Putative compounds that contact the nuclear receptor dimer/heterodimer regulatory site of nuclear receptor can also be designed. The overall folding of nuclear receptors based on a comparison of the reported structure of the unliganded RXR and with amino acid sequences of other superfamily members reveals that the overall folding of receptors of the superfamily is similar. Thus, by inspecting the three dimensional model of a protein or polypeptide that includes a DHRS, a putative agent (modulator) for the nuclear receptor can be designed. Steps include providing a three dimensional model of a protein or polypeptide that includes a nuclear receptor dimer/heterodimer regulatory site (DHRS) of the nuclear receptor of interest, and modeling the binding of one or more compounds to the three dimensional model/structure, thereby identifying one or more compound that binds to the DHRS. In one embodiment, the putative modulators can be tested for modulator activity as described herein and/or by methods known on one of skill in the art. Compounds produced by the method are also included in the present invention.

By “modeling” is intended quantitative and/or qualitative analysis of receptor structure/function based on three-dimensional structural information and receptor-compound interaction models. This includes conventional numeric-based molecular dynamic and energy minimization models, interactive computer graphic models, modified molecular mechanics models, distance geometry and other structure-based constraint models. Modeling is preferably performed using a computer and can be further optimized using known methods.

For example, computer programs such as DOCK, Catalyst, MCSS/Hook, etc., can be used to design one or more putative compounds that binds the DHRS. Other computer programs that use crystallography data can also be used to rationally design putative modulators of nuclear receptors. Programs such as RASMOL can be used with the atomic coordinates from crystals of nuclear receptors by generating three dimensional models and/or determining the structures involved in DHRS. Computer programs such as INSIGHT and GRASP allow for further manipulation and the ability to introduce new structures. Exemplary model ligands for use in the design of putative ligands (or in the assessment of a putative DHRS) include, but are not limited to, thyroid hormone analogs GC-1 and GC-24 (Table 2) and structures provided by Scanlan et al in U.S. Pat. No. 5,883,294 and U.S. Pat. No. 6,266,622, supra.

TABLE 2
THYROID HORMONE ANALOGS

| Compound name | Structure |
|---|---|
| GC-24 | 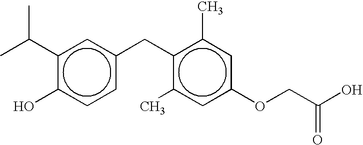 |
| GC-1 | 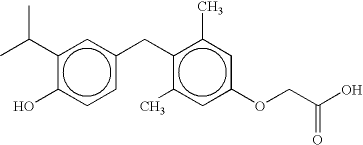 |
| Thyroid hormone (T₃) | 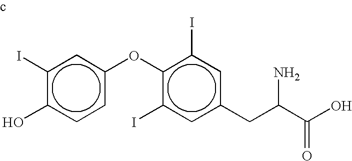 |

Optionally, a putative TR modulator can be designed by providing the atomic coordinates of a TR DHRS to a computerized modeling system, and modeling compounds which fit spatially into the TR DHRS. The putative modulators can then be identified in a biological assay for TR dimer/heterodimer formation, cofactor molecule interactions and/or TR receptor inactivation/activation.

Producing Agents

The modulatory compounds of the present invention can be obtained in a number of ways. They can be synthesized. Known compounds (including compound libraries) can be tested for putative modulator activity. Known compounds can also be modified to include a domain(s) that contacts the DHRS. A plurality of compounds can also be provided and modified by coupling a plurality of different domains to the plurality of compounds to provide a plurality of putative modulators of the invention. The putative modulators can be tested for modulator activity, selecting those that test positive for the desired activity.

Phage technology can also be used to provide test agents or agents of the present invention. See, e.g., Smith and Petrenko (1997) “Phage Display” Chem. Rev. 97:391-410. Using this technology, bacteriophage libraries that express, e.g., random peptide sequences that are presented on the surface of the phage particle (phage display) can be screened to isolate agents that contact a DHRS. Phage libraries and other types of libraries are described in the section herein entitled “Libraries of the Invention.” In certain embodiments, phage expressing desired molecules can be screen for those that modulate dimer/heterodimer function and/or cofactor molecule interactions or that occupy the DHRS. Phage can also be modeled. In certain embodiments, the modeled phage can be used to synthesize small organic molecules or to generate altered amino acids of the expressed peptide on the phage to improve the agent's ability to modulate nuclear receptor activation. See, e.g., Geistlinger and Guy (2001) “An inhibitor of the interaction of thyroid hormone receptor beta and glucocorticoid interacting protein 1” J. Am. Chem. Soc. 123:1525-1526.

Assays for Modulator Activity

The methods of this invention have immediate utility in screening for test agents, agents (modulators), etc. that modulate, e.g., dimer/heterodimer formation, nuclear receptor-cofactor molecule interactions, optionally including activation or inactivation of a nuclear receptor, e.g., in a container, in a cell, tissue or organism. The assays of this invention can be optimized for use in particular contexts, depending, for example, on the source and/or nature of the biological sample and/or the particular test agents or agents, and/or the analytic facilities available. Thus, for example, optimization can involve determining optimal conditions for binding assays, gel assays, fluorescence assays, chromatography assays, protein-protein interactions assays, co-immunoprecipitation assays, western blot assays, far western blot assays, fusion tag assays, capture assays, two-hybrid (e.g., yeast, mammalian, etc.) system assays, optimum sample processing conditions (e.g. preferred PCR conditions), hybridization conditions that maximize signal to noise, protocols that improve throughput, etc. In addition, assay formats can be selected and/or optimized according to the availability of equipment and/or reagents. Thus, for example, where antibodies or ELISA kits are available it can be desired to assay protein concentration or to assay protein-protein interaction via immunoprecipitations. Conversely, where it is desired to screen for an agent (modulators) that alter transcription of a nuclear receptor responsive gene or a nucleic acid (e.g., a reporter gene) having a nuclear receptor response element, nucleic acid based assays are preferred. Routine selection and optimization of assay formats is well known to those of ordinary skill in the art.

In certain embodiments, this invention provides methods of identifying, designing and producing agents (e.g., modulators) that modulate dimer/heterodimer formation and/or cofactor molecule interactions, as described herein. These agents can act to block dimer/heterodimer formation, thereby blocking activation of nuclear receptors. These agents can also act to interfere with interactions of a cofactor molecule (e.g., protein), e.g., a corepressor or coactivator, and a nuclear receptor. For example, an agent of the present invention can block or disassociate a corepressor from a nuclear receptor, thereby allowing activation of the receptor. The methods can involve confirming or testing, e.g., by screening, an agent for activity that modulates the effect(s), e.g., as described herein (e.g., modulator activity), of a nuclear receptor, e.g., in vitro, or in vivo, or a combination of both.

In one embodiment, this includes binding an agent to the DHRS and testing the resulting agent-bound nuclear receptor for alterations of a protein exposed to the agent and/or detecting cellular events associated (e.g. dimer/heterodimer formation, alterations in interactions of a nuclear receptor and a cofactor molecule, receptor activation, receptor inactivation, other protein associations, protein conformational changes, protein modification, gene expression etc.) with the agent. In certain embodiments, the screening methods of this invention can involve contacting a protein or polypeptide comprising a DHRS (or a test cell, e.g., mammalian cell, that contains a protein or polypeptide comprising a DHRS, or a test organism, e.g., a mammalian organism, that contains a protein or polypeptide comprising a DHRS) with a test agent (e.g., a putative modulator, or an modulator depending on the application); and detecting alterations of a protein exposed to the test agent and/or detecting cellular events associated with the test agent. In another embodiment, testing includes binding a plurality of putative modulators to the nuclear receptor, selecting for members of the plurality of putative modulators that bind the DHRS, and testing the resulting bound nuclear receptors for modulator activity (e.g., dimer/heterodimer formation, alterations in interactions of a nuclear receptor and a cofactor molecule, receptor activation, receptor inactivation, other protein associations, protein conformational changes, protein modifications, gene expression etc.).

Protein Associations

For example, alterations of dimer/heterodimer formation of a nuclear receptor, alterations of nuclear receptor cofactor molecule interactions, or other protein associations via the DHRS, can be determined by, e.g., assays to determine protein complexes, alterations of dissociation of one or more cofactor molecules, e.g., transcriptional repressor proteins, from the nuclear receptor, alterations of dissociation of a heat shock protein from the nuclear receptor, etc.

Assays to determine protein complexes with a DHRS include, but are not limited to, co-immunoprecipitation, cross-linking and co-purification through gradients or chromatographic columns, gel-shift assays, western blots, far western blots, fusion tag assays, capture assays, e.g., using a normatural amino acid, two-hybrid (e.g., yeast, mammalian, etc.) systems, etc., described herein, and other known in the art. In addition, a variety of protein methods are well known in the art, including, e.g., those set forth in, e.g., Bollag et al. (1996) Protein Methods, 2nd Edition Wiley-Liss, NY; Walker (2002) The Protein Protocols Handbook, 2^{nd} Edition Humana Press, NJ, Harris; Walker (2002) Protein Protocols on CD-ROM Humana Press, NJ; and, Golemis, (2001) Protein-Protein Interactions: A Molecular Cloning Manual, Cold Spring Harbor Laboratory, NY, and the references cited therein.

Alternations in protein interactions can be measured by various different methods know to one of skill in the art, including, but not limited to, gel shift assays, fluorescence assays, chromatography assays, etc. Other suitable assays are described herein and in, e.g., Shibata et al. (1997) Recent Prog. Horm. Res. 52:141-164; Tagami et al. (1997) Mol. Cell Biol. 17(5):2642-2648; Zhu et al. (1997) J. Biol. Chem. 272(14):9048-9054; Lin et al. (1997) Mol. Cell Biol. 17(10):6131-6138; Kakizawa et al. (1997) J. Biol. Chem. 272(38):23799-23804; and Chang et al. (1997) Proc. Natl. Acad. Sci. USA 94(17):9040-9045. For example, high throughput binding and bioactivity assays can be devised using purified recombinant protein and modern reporter gene transcription assays described herein and known in the art in order to confirm, test, etc. for activity. Agents of the present invention can affect one or more of these activities.

Nuclear receptors or nuclear receptor LBDs usually have activation domains modulated in part by a coactivator/corepressor system that coordinately functions to present a region for binding to DNA, and that can be modulated by the binding of an agent of the invention to the DHRS. For example, receptors that are not associated with hsp in the absence of ligand can act as transcriptional repressors of positively regulated genes in the absence of the ligand. This appears to be due, in part, to transcriptional repressor proteins that bind to the LBD of the receptors. Agonist binding induces a dissociation of these proteins from the receptors. This relieves the inhibition of transcription and allows the transcriptional transactivation functions of the receptors to become manifest. Unliganded receptors that are not associated with hsp can also activate gene transcription in many contexts. Here, an agent of the invention can reverse the positive effect of unliganded receptor and can suppress receptor activity by blocking or dissociating a corepressor from interaction with the nuclear receptor, and/or promoting binding or association of a coactivator. Alternatively, an agent of the invention can also block coactivator interaction and/or promote corepressor interaction with the nuclear receptor.

Dissociation of a heat shock protein from the nuclear receptor can also be used for assaying for modulation of dimer/heterodimer formation, modulation of cofactor molecule interactions, and/or modulation of nuclear receptor activation. For many of the nuclear receptors ligand binding induces a dissociation of heat shock proteins such that the receptors can form dimers in most cases, after which the receptors bind to DNA and regulate transcription. Nuclear receptors usually have heat shock protein binding domains that present a region for binding to the LBD and can be modulated by the binding of an agent of the invention. Consequently, an agent of the invention can stabilize or destabilize the binding or contact of the heat shock protein with the LBD.

The agents of the invention can also modulate a receptor's interaction with other proteins involved in transcription. These could be proteins that interact directly or indirectly with elements of the proximal promoter or proteins of the proximal promoter. Alternatively, the interactions could be through other transcription factors that themselves interact directly or indirectly with proteins of the proximal promoter. In addition, it is possible that in some cases, agent-induced conformational changes do not affect the binding of other proteins to the receptor, but do affect their abilities to regulate transcription. These activities can be detected used gene expression assays described herein and other assays known to one of skill in the art.

Conformational Changes

Modulation of the nuclear receptor via the DHRS can also be confirmed or tested by using assays that examine conformational changes in the receptor, e.g., due to dimerization/heterodimerization, cofactor molecule interactions, etc. An agent-free receptor can be compared to a nuclear receptor with bound agent using conventional techniques. For example, a column can be used that separates the receptor according to charge, such as an ion exchange or hydrophobic interaction column.

In addition, various conformations of receptors can also be assessed by phage technology. With this technology, bacteriophage libraries that express random peptide sequences that are presented on the surface of the phage particle (Phage display) can be screened to isolate peptides that recognize individual conformational states of receptors. Thus, phage can be isolated that express peptides that distinguish between different forms of the receptor, receptors in various states of transcriptional activation, etc. Such phage can then be used to screen libraries of compounds for the requisite conformation. With respect to the current invention, this would be conformations that reflect, e.g., dimer/heterodimer formation of the receptor, interactions of a nuclear receptor and a cofactor molecule, the state of activation of the receptor, etc. See, e.g., Wijayaratne et al. (1999) Endocrinology. 140:5828; Chang et al. (1999) Mol Cell Biol 19:8226; Norris et al. (1999) Science 285:744; and, Paige et al. (1999) PNAS 96:3999.

Cellular Location of a Nuclear Receptor Exposed to an Agent

Modulation of nuclear receptor activation (e.g., due to modulation of dimer/heterodimer formation and/or cofactor molecule interactions) can also be identified by assaying for the cellular location of a nuclear receptor. Using the techniques described herein, one of skill in the art can identify nuclear receptors that enter (or don't enter) the nucleus as an indication of nuclear activation (or inactivation). The localization of proteins can be determined in a variety of ways as described below. Generally, cells are examined for evidence of (1) a decrease in the amount of the protein in an origin cellular subregion; (2) an increase in the amount of the protein in a destination cellular subregion (or in an intermediate destination cellular subregion); or (3) a change in the distribution of the protein in the cellular subregions of the cell. The evidence can be direct or indirect. An example of indirect evidence is the detection of a cellular event mediated by the protein including, but not limited to, the cellular events discussed below.

Detecting Subcellular Distribution of a Protein.

Determination of the localization of the nuclear receptor (or proteins modulated by the state (activated or inactivated) of the nuclear receptor) can be carried out in any of a number of ways. A preferred way is by detection of a colorimetric change, for example, by visual observation. Various methods of visual observation can be used, such as light microscopy, fluorescence microscopy, and confocal microscopy. If desired, an epifluorescence microscope with a CCD camera can be used to measure translocation in the assays described below. This procedure can be automated, for example, by computer-based image recognition. The intracellular distribution of the protein can be determined by staining a cell with a stain specific for the protein. The stain comprises a specific binding substance, which binds specifically to the targeted protein. Examples of such a stain include, but are not limited to, antibodies that specifically bind to the protein. A stain specific for, e.g., a nuclear receptor can be prepared using known immunocytochemistry techniques. Stains specific for other proteins having cellular locations or quantities that can be correlated with nuclear receptor activation can be similarly prepared. Preferably, the stain further comprises a labeling moiety. Suitable antibodies can be prepared using conventional antibody production techniques. The antibodies can be monoclonal or polyclonal. Antibody fragments, such as, for example Fab fragments, Fv fragments, and the like, are also contemplated. The antibodies can also be obtained from genetically engineered hosts or from conventional sources. Techniques for antibody production are well known to the person of ordinary skill in the art and examples of such techniques can be found in Harlow and Lane, Antibodies: A Laboratory Manual, Cold Spring Harbor Laboratory Press, Cold Spring Harbor (1988), Birch and Lennox, Monoclonal Antibodies: Principles and Applications, Wiley-Liss, New York (1995). The labeling moiety will be visibly observable in conventional immunohistochemical detection techniques being, for example, a fluorescent dye such as fluorescein, a chemiluminescense reagent, a radioisotope, a colloidal label, such as colloidal gold or colored latex beads, an enzyme label, or any other known labeling complex. Such stains can be prepared by conventional techniques, for example as described in Manson (1992) Immunochemical Protocols: Methods in Molecular Biology Vol. 10, Humana Press, Totowa, N.J., and Beesley (1993) Immunocytochemistry: A Practical Approach, IRL Press, Oxford, England.

Fusion proteins can also be used to track the localization of a protein. The fusion partner can be detectable directly, such as the green fluorescent protein (GFP), or can be detected indirectly using antibodies specific for the fusion partner or by detecting the enzymatic products of a fusion partner such as β-galactosidase. Cells, which express a fusion protein, can be prepared by transfecting a host cell with a polynucleotide encoding the fusion protein. Preferably, the fusion protein is expressed at levels low enough to avoid expression in vast excess of other cellular factors, which can be required for subcellular localization of the protein. For example, if a 100-fold molar excess of the fusion protein is expressed relative to a factor required for translocation from the origin subregion to the destination subregion, translocation upon exposure to, e.g., a modulator, cannot be detectable because most of the fusion protein would remain unbound in the origin subregion. This goal can be achieved by not using strong promoters, enhancers or origins of replication giving rise to high copy numbers of plasmids, and by transfecting with smaller amounts of DNA. Preferred fusion proteins include GFP fused to a protein for which its localization is of interest, such as, for example, nuclear receptor. GFP can be fused to either the amino terminus or the carboxy terminus of the protein of interest. A tag, such as a histidine tag, can be included, if desired.

Another preferred way to detect a colorimetric change is to use more than one stain. Preferably, the combination of the stains results in a different color than either stain alone. For example, a cell can be stained with a first stain specific for a particular cellular subregion to be examined and a second stain specific for a particular state (activated or inactivated) of a nuclear receptor or indicative protein that migrates to or from that cellular subregion in a cell exposed to an agent. Examples of such staining systems are known in the art and can be adapted for use in the methods described below. A preferred staining system involves the use of a fluorescence indicator, such as, for example, fluorescein, Cy3, Cy5, Texas Red, rhodamine, and the like. For example, modulator-treated cells can be stained with antibodies to nuclear receptor and secondary antibodies conjugated to fluorescein, which would stain the nuclei green. If the cells are further stained with a red nuclear-specific dye (such as, for example, TOTO-3), the nuclei with nuclear receptor will appear yellow instead of red. Other dyes for specific cellular subregions include, but are not limited to, Golgi markers such as mannosidase II and BODIPY TR ceramide (Molecular Probes), nuclear markers such as Neu N, and conjugated antibodies recognizing proteins specific to a particular subregion such as Golgi marker enzymes, histones, and the like.

The particular protein and cellular subregion(s) selected for examination can vary depending on the cell type to be used in a particular method. In one embodiment, cells used in the methods of the invention are of a cell type in which the selected protein is predominantly present in a different amount in a particular cellular subregion of modulator-exposed cells compared to modulator-unexposed cells.

Modification.

Besides a change in protein associations (or the other cellular events described herein) of a protein exposed to an agent, an agent of the invention can trigger other cellular events that can be detected, e.g., phosphorylation, ubiquitination, SUMOlation (SUMO—small ubiquitin-related modifier), acetylation, etc. Another aspect of the invention is to provide methods for detecting the effects of an agent's modulation of a state (activated or inactivated) of a nuclear receptor on cells by measuring the modification of proteins that are differentially modified in the presence or absence of an agent of the invention.

The identity of proteins that are differentially modified in response to the agent of the invention can readily be determined using conventional assay techniques known to the person of skill in the art. For example, radioactively labeled molecules, e.g., phosphate, can be added to cultured cells grown in both the presence and absence of agent. Proteins from the labeled cells can then be extracted and separated on a one or two dimensional gel system. Isolated modified proteins can then be visualized by autoradiography and related techniques. After separation and visualization, changes in the level of modification of different proteins can be determined by comparing the results obtained from cells exposed to a modulator with the results obtained from cells not exposed to a modulator. Preferably, proteins of interest are immunoprecipitated. Proteins that are differentially modified can be identified by amino terminus amino acid residue sequencing.

A more sensitive detection method involves the use of antibodies, for example, antibodies that recognize phosphorylated forms of specific proteins, or antibodies that recognize a phosphorylated amino acid residue, such as phosphothreonine or phosphoserine antibodies. Another useful detection method for phosphorylation modification is back-phosphorylation, which is safer than direct phosphorylation assays but less sensitive. Cell extracts are incubated with radiolabeled ATP and Mg⁺² and subjected to gel electrophoresis. Since a modulator can alter phosphorylation, a different amount of radiolabeled phosphate will be incorporated into individual proteins of cells exposed to the modulator than in cells that have not been so exposed, resulting in a different pattern of bands on a gel.

Proteins that are differentially modified in response to receptor activation can be used in assays for the exposure of cells to a modulator. Furthermore, these differentially modified proteins can be used as the targets when screening for compounds that modulate the cellular effects of a modulator that contacts a DHRS of a nuclear receptor. Such assays include assays involving the steps of measuring the modification of differentially modified proteins. Compounds could be screened by measuring their effects on modification of these differentially modified proteins.

Gene Expression

An agent of the invention can affect gene regulation, either directly or indirectly. For purposes of the methods described below, the gene is regulated by a nuclear receptor (whether directly or indirectly). Thus, alterations in transcription of a nuclear responsive gene (NRRG) and/or a nucleic acid comprising a nuclear receptor responsive element operably linked to, e.g., a reporter gene, can be used for screening for agents that contact the DHRS, assaying modulation of dimer/heterodimer formation, assaying modulation of interactions of a nuclear protein and a cofactor molecule, assaying modulation of activation of a nuclear receptor, etc. For example, agents of the invention modulate dimer/heterodimer formation. Thus, by, e.g., blocking dimer/heterodimer formation, agents of the invention can modulate nuclear receptor DBD binding to DNA. Consequently, an agent of the invention can influence DNA transcription by modulating dimer/heterodimer formation of a nuclear receptor. In certain embodiments, these activities can be assayed by DNA binding and/or transcription of, e.g., a NRRG or a reporter gene linked to a nuclear receptor responsive element. Alternatively, an agent of the invention, which blocks dimer/heterodimer formation, can disrupt interactions with other proteins involved in transcriptional regulation, thereby modulating the protein ability to bind to DNA. These activities can be assay by DNA binding and/or transcription of a gene which is dependent on the protein for transcriptional regulation, or by a responsive element operably linked to a reporter gene.

For example, in nuclear receptors that bind to heat shock protein (hsp), the ligand-induced dissociation of hsp with consequent dimer formation allows, and therefore promotes, DNA binding. With receptors that are not associated with hsp (as in the absence of ligand), ligand binding can either stimulate or discourage DNA binding of homodimers, and increase monomer binding to DNA. Binding of most nuclear receptors to DNA involves use of 2 half sites, each of which is the binding site for one of each pair of the homodimer or heterodimer. In these cases, for example, an agent of the invention which blocks hsp dissociation, or blocks homodimer/heterodimer formation greatly decreases DNA binding of the receptors to the element.

In certain embodiments, the screening methods can involve detecting the expression or activity of a nuclear receptor responsive gene (NRRG) or of a reporter gene (RG) linked to a responsive element of interest of said test cell wherein a difference in NRRG or RG expression or activity in said test cell as compared to nuclear receptor responsive gene or reporter gene expression or activity in a control cell indicates that said test agent modulates dimer/heterodimer formation or cofactor molecule interactions with the nuclear receptor.

Expression levels of a gene can be altered by changes in the transcription of the gene product (i.e. transcription of mRNA), and/or by changes in translation of the gene product (i.e. translation of the protein), and/or by post-translational modification(s) (e.g. protein folding, glycosylation, etc.). Assays of this invention include assaying for level of transcribed mRNA (or other nucleic acids derived from nucleic acids that encode a polypeptide comprising a nuclear receptor responsive gene), level of translated protein, activity of translated protein, etc. Examples of such approaches are described below. These examples are intended to be illustrative and not limiting.

Gene transcription modulated by an agent that modulates dimer/heterodimer formation or cofactor molecule interactions of nuclear receptors can be monitored by assays known to one of skill in the art and those described herein. For example, at least one nuclear receptor responsive gene (NRRG) and/or a nuclear receptor response element, e.g., thyroid hormone response element (TRE), glucocorticoid hormone response element (GRE), etc., can be coupled with a reporter gene, the expression of which is controlled by an activated nuclear receptor. Alternatively, a desired response element (e.g., an element that interacts with a protein, which is modulated by a nuclear receptor but is other than a nuclear receptor) can be coupled with a reported gene, the expression of which is controlled via the nuclear receptor. In certain embodiments, control of expression by activated nuclear receptor can be enhanced by increasing the number of binding sites for an activated nuclear receptor or for the protein in the vicinity of the reporter gene. Examples of reporter genes, include, but are not limited to chloramphenicol acetyl transferase (CAT) (Alton et al., Nature (1979) 282:864-869), beta-galactosidase, firefly luciferase (deWet et al., Mol. Cell. Biol. (1987) 7:725-737), bacterial luciferase (Engebrecht et al., Proc. Natl. Acad. Sci. USA (1984) 1:4154-4158; Baldwin et al., Biochemistry (1984) 23:3663-3667, alkaline phosphatase (Toh et al., J. Biochem. (1989) 182:231-238; Hall et al., J. Mol. Appl. Gen. (1983) 2:101, and green fluorescent protein (GFP) (Meyer et al., Diabetes (1998) 47(12):1974-1977), a GFP-luciferase fusion protein (Day et al. Biotechniques 1998 25(5):848-850, 852-854, 856), and other genes encoding a detectable gene product. Detection of gene expression can be achieved in a variety of ways depending on the reporter gene used. For example, a fluorescence or chemiluminescence detection system can be used to detect expression of luciferase and GFP. A nuclear receptor response element-dependent GFP construct can be used. Alternatively, an antibody that recognizes the gene product encoded by a reporter gene can be used to detect expression of many reporter genes as well as many endogenous genes regulated by nuclear receptors. Visual observation of a colorimetric change can be used to detect expression of beta-galactosidase or alkaline phosphatase. A reporter gene can be inserted into the cells by various techniques known in the art and described herein. Transient expression is preferred. However, the reporter gene can be present on a vector that is stably integrated into the genome of the cells.

The expression of genes can be monitored by any of a number of ways known in the art and described herein, such as, for example, by Northern analysis, polymerase chain reaction (PCR), Western analysis, radioimmunoassays (RIA), enzyme linked immunoassays (ELISA or EIA), fluorescence activated cell sorting (FACS) analysis, enzyme-substrate assays such as chloramphenicol transferase (CAT) assays, and the like. Preferably, expression of such genes in response to a modulator binding the DHRS is determined by detecting a difference in a signal that is at least about 1.5 times that of control cells which have not been exposed to the modulator, preferably greater than about 2×, and often 10× or more.

Nucleic-Acid Based Assays.

Target Molecules.

Changes in expression levels of a gene can be detected by measuring changes in mRNA and/or a nucleic acid derived from the mRNA (e.g. reverse-transcribed cDNA, etc.) that encodes a polypeptide of the gene product of NRRG, a gene product of a nucleic acid that has a nuclear responsive element or a gene product of a nucleic acid that has a desired responsive element. In order to measure the gene expression level, it is desirable to provide a nucleic acid sample for such analysis. In preferred embodiments, the nucleic acid is found in or derived from a biological sample. The term “biological sample”, as used herein, refers to a sample obtained from an organism or from components (e.g., cells) of an organism, or of a cell or of a tissue culture.

The nucleic acid (e.g., mRNA nucleic acid derived from mRNA) is, in certain preferred embodiments, isolated from the sample according to any of a number of methods well known to those of skill in the art. Methods of isolating mRNA are well known to those of skill in the art. For example, methods of isolation and purification of nucleic acids are described in detail in by Tijssen ed., (1993) Chapter 3 of Laboratory Techniques in Biochemistry and Molecular Biology: Hybridization With Nucleic Acid Probes, Part I. Theory and Nucleic Acid Preparation, Elsevier, N.Y. and Tijssen ed.

In a preferred embodiment, the “total” nucleic acid is isolated from a given sample using, for example, an acid guanidinium-phenol-chloroform extraction method and polyA+ mRNA is isolated by oligo dT column chromatography or by using (dT)ₙ magnetic beads (see, e.g., Sambrook et al., Molecular Cloning: A Laboratory Manual (3rd ed.), Vols. 1-3, Cold Spring Harbor Laboratory, (2001), or Current Protocols in Molecular Biology, F. Ausubel et al., ed. Greene Publishing and Wiley-Interscience, New York (1997 and supplemented through 2002)).

Frequently, it is desirable to amplify the nucleic acid sample prior to assaying for expression level. Methods of amplifying nucleic acids are well known to those of skill in the art and include, but are not limited to polymerase chain reaction (PCR, see, e.g., Innis, et al., (1990) PCR Protocols. A guide to Methods and Application. Academic Press, Inc. San Diego,), ligase chain reaction (LCR) (see Wu and Wallace (1989) Genomics 4: 560, Landegren et al. (1988) Science 241: 1077, and Barringer et al. (1990) Gene 89: 117), transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.).

In one embodiment, where it is desired to quantify the transcription level (and thereby expression) of NRRG or RG in a sample, the nucleic acid sample is one in which the concentration of the NRRG mRNA transcript(s), or the concentration of the nucleic acids derived from the NRRG polypeptide mRNA transcript(s), is proportional to the transcription level (and therefore expression level) of that gene. Similarly, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Thus, for example, an assay where a 5 fold difference in concentration of the target mRNA results in a 3 to 6 fold difference in hybridization intensity is sufficient for most purposes.

Where more precise quantification is required, appropriate controls can be run to correct for variations introduced in sample preparation and hybridization as described herein. In addition, serial dilutions of “standard” target nucleic acids (e.g., mRNAs) can be used to prepare calibration curves according to methods well known to those of skill in the art. Of course, where simple detection of the presence or absence of a transcript or large differences of changes in nucleic acid concentration is desired, no elaborate control or calibration is required.

In the simplest embodiment, the sample comprises a nucleic acid comprising a NRRG encoded polypeptide in the total mRNA or a total cDNA isolated and/or otherwise derived from a biological sample. The nucleic acid can be isolated from the sample according to any of a number of methods well known to those of skill in the art as indicated above.

Hybridization-Based Assays.

Using the known nucleic acid sequences encoding polypeptides encoded by NRRG or RG, detecting and/or quantifying transcript(s) of these nucleic acids can be routinely accomplished using nucleic acid hybridization techniques (see, e.g., Sambrook et al. supra). For example, one method for evaluating the presence, absence, or quantity of reverse-transcribed cDNA involves a “Southern Blot.” Alternatively, the mRNA can be directly quantified in a Northern blot. An alternative means for determining the NRRG expression level is in situ hybridization. In situ hybridization assays are well known (e.g., Angerer (1987) Meth. Enzymol 152: 649). The reagent used in in situ hybridization assays and the conditions for use vary depending on the particular application. In some applications, it is necessary to block the hybridization capacity of repetitive sequences. Thus, in some embodiments, tRNA, human genomic DNA, or Cot-1 DNA is used to block non-specific hybridization.

Amplification-Based Assays.

In another embodiment, amplification-based assays can be used to measure NRRG expression (transcription) level or the reporter gene constructs described herein. In such amplification-based assays, the target nucleic acid sequences (e.g., a nucleic acid comprising a NRRG encoded polypeptide or fragment thereof) act as template(s) in amplification reaction(s) (e.g. Polymerase Chain Reaction (PCR) or reverse-transcription PCR (RT-PCR)). In a quantitative amplification, the amount of amplification product will be proportional to the amount of template (e.g., NRRG polypeptide-encoding mRNA) in the original sample. Comparison to appropriate (e.g. healthy tissue or cells unexposed to the test agent) controls provides a measure of the transcript level.

Methods of “quantitative” amplification are well known to those of skill in the art. For example, quantitative PCR involves simultaneously co-amplifying a known quantity of a control sequence using the same primers. This provides an internal standard that can be used to calibrate the PCR reaction. Detailed protocols for quantitative PCR are provided in Innis et al. (1990) PCR Protocols, A Guide to Methods and Applications, Academic Press, Inc. N.Y.). One approach, for example, involves simultaneously co-amplifying a known quantity of a control sequence using the same primers as those used to amplify the target. This provides an internal standard that can be used to calibrate the PCR reaction.

Real time PCR and/or RT-PCR (e.g., mediated via TaqMan™ probes (operating by detecting a double-labeled probe before, during, or after polymerase-mediated digestion of the double labeled probe) or molecular beacon-based probes) can also be used to facilitate detection of amplified nucleic acids. Real time detection can be omitted, e.g., simply by detecting amplicons via labeled probes, e.g., after separation of the amplicon from unlabeled probe. Further details regarding TaqMan™ are found in, e.g., U.S. Pat. No. 5,487,972; Mackay et al. (2002) Nucl Acids Res 30:1292-1305, and references cited within. Molecular beacons (MBs) are oligonucleotides, which can be comprised of standard or modified nucleotides or analogs thereof (e.g., peptide nucleic acids (PNAs)), designed for the detection and quantification of target nucleic acids (e.g., target DNAs). MBs are gaining wide spread acceptance as robust reagents for detecting and quantitating nucleic acids, including in real time (e.g., MBs can be used to detect targets as they are formed, e.g., by PCR). A variety of commercial suppliers produce standard and custom molecular beacons, including Cruachem (cruachem.com), Oswel Research Products Ltd. (UK; oswel.com), Research Genetics (a division of Invitrogen, Huntsville Ala. (resgen.com)), the Midland Certified Reagent Company (Midland, Tex. mcrc.com) and Gorilla Genomics, Inc. (Alameda, Calif.).

Further details regarding methods of MB manufacture and use are found, e.g., in Leone et al. (1995) “Molecular beacon probes combined with amplification by NASBA enable homogenous real-time detection of RNA” Nucleic Acids Res. 26:2150-2155; Tyagi and Kramer (1996) “Molecular beacons: probes that fluoresce upon hybridization” Nature Biotechnology 14:303-308; Blok and Kramer (1997) “Amplifiable hybridization probes containing a molecular switch” Mol Cell Probes 11: 187-194; Hsuih et al. (1997) “Novel, ligation-dependent PCR assay for detection of hepatitis C in serum” J Clin Microbiol 34:501-507; Kostrikis et al. (1998) “Molecular beacons: spectral genotyping of human alleles” Science 279:1228-1229; Sokol et al. (1998) “Real time detection of DNA:RNA hybridization in living cells” Proc. Natl. Acad. Sci. U.S.A. 95:11538-11543; Tyagi et al. (1998) “Multicolor molecular beacons for allele discrimination” Nature Biotechnology 16:49-53; Bonnet et al. (1999) “Thermodynamic basis of the chemical specificity of structured DNA probes” Proc. Natl. Acad. Sci. U.S.A. 96:6171-6176; Fang et al. (1999) “Designing a novel molecular beacon for surface-immobilized DNA hybridization studies” J. Am. Chem. Soc. 121:2921-2922; Marras et al. (1999) “Multiplex detection of single-nucleotide variation using molecular beacons” Genet. Anal. Biomol. Eng. 14:151-156; and, Vet et al. (1999) “Multiplex detection of four pathogenic retroviruses using molecular beacons” Proc. Natl. Acad. Sci. U.S.A. 96:6394-6399. Kits utilizing TaqMan™ probes and/or molecular beacons are commonly available for performing real time PCR analysis, and can be used for these applications in the present invention.

Hybridization Formats and Optimization of Hybridization Conditions.

a) Array-Based Hybridization Formats.

In one embodiment, the methods of this invention can be utilized in array-based hybridization formats. Arrays have a multiplicity of different “probe” or “target” nucleic acids (or other compounds), e.g., attached to one or more surfaces (e.g., solid, membrane, or gel). In a preferred embodiment, the multiplicity of nucleic acids (or other moieties) is attached to a single contiguous surface or to a multiplicity of surfaces juxtaposed to each other. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211). See also U.S. Pat. No. 5,807,522, U.S. Pat. No. 5,143,854, U.S. Pat. No. 5,744,305, U.S. Pat. No. 5,744,305 U.S. Pat. No. 5,800,992, U.S. Pat. No. 5,445,934 and PCT Patent Publication Nos. WO 90/15070 and 92/10092

b) Other Hybridization Formats.

As indicated above, a variety of nucleic acid hybridization formats are known to those skilled in the art. For example, common formats include sandwich assays and competition or displacement assays. Such assay formats are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587. Typically, labeled signal nucleic acids are used to detect hybridization. Complementary nucleic acids or signal nucleic acids can be labeled by any one of several methods typically used to detect the presence of hybridized polynucleotides as described herein.

The sensitivity of the hybridization assays can be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBA, Cangene, Mississauga, Ontario) and Q Beta Replicase systems.

c) Optimization of Hybridization Conditions.

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization requires fewer mismatches.

One of skill in the art will appreciate that hybridization conditions can be selected to provide any degree of stringency. Hybridization specificity can be evaluated by comparison of hybridization to the test probes with hybridization to the various controls that can be present.

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array can be washed at successively higher stringency solutions and read between each wash. Analysis of the data sets thus produced will reveal a wash stringency above which the hybridization pattern is not appreciably altered and which provides adequate signal for the particular probes of interest.

Optionally, background signal is reduced by the use of a blocking reagent (e.g., tRNA, sperm DNA, cot-1 DNA, etc.) during the hybridization to reduce non-specific binding. The use of blocking agents in hybridization is well known to those of skill in the art (see, e.g., Chapter 8 in P. Tijssen, supra.)

Methods of optimizing hybridization conditions are well known to those of skill in the art (see, e.g., Tijssen (1993) Laboratory Techniques in Biochemistry and Molecular Biology, Vol. 24: Hybridization With Nucleic Acid Probes, Elsevier, N.Y.).

Optimal conditions are also a function of the sensitivity of label (e.g., fluorescence) detection for different combinations of substrate type, fluorochrome, excitation and emission bands, spot size and the like. Low fluorescence background surfaces can be used (see, e.g., Chu (1992) Electrophoresis 13:105-114). The sensitivity for detection of spots (“target elements”) of various diameters on the candidate surfaces can be readily determined by, e.g., spotting a dilution series of fluorescently end labeled DNA fragments. These spots are then imaged using conventional fluorescence microscopy. The sensitivity, linearity, and dynamic range achievable from the various combinations of fluorochrome and solid surfaces (e.g., glass, fused silica, etc.) can thus be determined. Serial dilutions of pairs of fluorochrome in known relative proportions can also be analyzed. This determines the accuracy with which fluorescence ratio measurements reflect actual fluorochrome ratios over the dynamic range permitted by the detectors and fluorescence of the substrate upon which the probe has been fixed.

d) Labeling and Detection of Nucleic Acids.

The probes used herein for detection of gene expression levels can be full length or less than the full length of the polypeptides comprising the gene encoded protein. Shorter probes are empirically tested for specificity. Preferred probes are sufficiently long so as to specifically hybridize with the target nucleic acid(s) under stringent conditions. The preferred size range is from about 20 bases to the length of the target mRNA, more preferably from about 30 bases to the length of the target mRNA, and most preferably from about 40 bases to the length of the target mRNA.

The probes are typically labeled, with a detectable label. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, Texas red, rhodamine, green fluorescent protein, and the like, see, e.g., Molecular Probes, Eugene, Oreg., USA), radiolabels (e.g., ³H, ²⁵I, ³⁵S, ¹⁴C, or ³²P), enzymes (e.g., horse radish peroxidase, alkaline phosphatase and others commonly used in an ELISA), and calorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

A fluorescent label is preferred because it provides a very strong signal with low background. It is also optically detectable at high resolution and sensitivity through a quick scanning procedure. The nucleic acid samples can all be labeled with a single label, e.g., a single fluorescent label. Alternatively, in another embodiment, different nucleic acid samples can be simultaneously hybridized where each nucleic acid sample has a different label. For instance, one target could have a green fluorescent label and a second target could have a red fluorescent label. The scanning step will distinguish sites of binding of the red label from those binding the green fluorescent label. Each nucleic acid sample (target nucleic acid) can be analyzed independently from one another.

Suitable chromogens that can be employed include those molecules and compounds which absorb light in a distinctive range of wavelengths so that a color can be observed or, alternatively, which emit light when irradiated with radiation of a particular wave length or wave length range, e.g., fluorescers.

Detectable signal can also be provided by chemiluminescent and bioluminescent sources. Chemiluminescent sources include a compound that becomes electronically excited by a chemical reaction and can then emit light which serves as the detectable signal or donates energy to a fluorescent acceptor. Alternatively, luciferins can be used in conjunction with luciferase or lucigenins to provide bioluminescence.

Spin labels are provided by reporter molecules with an unpaired electron spin, which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

The label can be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid can be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

Fluorescent labels are easily added during an in vitro transcription reaction. Thus, for example, fluorescein labeled UTP and CTP can be incorporated into the RNA produced in an in vitro transcription.

The labels can be attached directly or through a linker moiety. In general, the site of label or linker-label attachment is not limited to any specific position. For example, a label can be attached to a nucleoside, nucleotide, or analogue thereof at any position that does not interfere with detection or hybridization as desired. For example, certain Label-ON Reagents from Clontech (Palo Alto, Calif.) provide for labeling interspersed throughout the phosphate backbone of an oligonucleotide and for terminal labeling at the 3′ and 5′ ends. As shown for example herein, labels can be attached at positions on the ribose ring or the ribose can be modified and even eliminated as desired. The base moieties of useful labeling reagents can include those that are naturally occurring or modified in a manner that does not interfere with the purpose to which they are put. Modified bases include but are not limited to 7-deaza A and G, 7-deaza-8-aza A and G, and other heterocyclic moieties.

It will be recognized that fluorescent labels are not to be limited to single species organic molecules, but include inorganic molecules, multi-molecular mixtures of organic and/or inorganic molecules, crystals, heteropolymers, and the like. Thus, for example, CdSe-CdS core-shell nanocrystals enclosed in a silica shell can be easily derivatized for coupling to a biological molecule (Bruchez et al. (1998) Science, 281: 2013-2016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018).

Polypeptide-Based Assays.

Assay Formats

In addition to, or in alternative to, the detection of nucleic acid expression level(s), alterations in expression or activity of a NRRG encoded protein or a reporter gene can be detected and/or quantified by detecting and/or quantifying the amount and/or activity of a translated NRRG or reporter gene encoded polypeptide.

Detection of Expressed Protein

The polypeptide(s) comprising a NRRG or the reporter gene encoded protein can be detected and quantified by any of a number of methods well known to those of skill in the art. These can include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like.

In one embodiment, a NRRG or reporter gene encoded polypeptide is detected/quantified in an electrophoretic protein separation (e.g. a 1- or 2-dimensional electrophoresis). Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.; Sandana (1997) Bioseparation of Proteins, Academic Press, Inc.; Bollag et al. (1996) Protein Methods, 2^{nd} Edition Wiley-Liss, NY; Walker (2002) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, NY; Janson and Ryden (1998) Protein Purification: Principles, High Resolution Methods and Applications, Second Edition Wiley-VCH, NY; and Walker (2002) Protein Protocols on CD-ROM Humana Press, NJ; and the references cited therein). In another preferred embodiment, Western blot (immunoblot) analysis is used to detect and quantify the presence of a NRRG encoded protein. Many other applicable methods are described in Walker (2002), herein.

The antibodies specifically bind to the target polypeptide(s) and can be directly labeled or alternatively can be subsequently detected using labeled antibodies (e.g., labeled sheep anti-mouse antibodies) that specifically bind to a domain of the antibody.

In certain embodiments, a NRRG or reporter gene encoded polypeptide is detected using an immunoassay. As used herein, an immunoassay is an assay that utilizes an antibody to specifically bind to the analyte (e.g., the target polypeptide(s)). The immunoassay is thus characterized by detection of specific binding of a polypeptide of this invention to an antibody as opposed to the use of other physical or chemical properties to isolate, target, and quantify the analyte.

Any of a number of well recognized immunological binding assays (see, e.g., U.S. Pat. Nos. 4,366,241; 4,376,110; 4,517,288; and 4,837,168) are well suited to detection or quantification of the polypeptide(s) identified herein. For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (NRRG or reporter gene encoded polypeptide(s)). In preferred embodiments, the capture agent is an antibody.

Immunoassays also often utilize a labeling agent to specifically bind to and label the binding complex formed by the capture agent and the analyte. The labeling agent can itself be one of the moieties comprising the antibody/analyte complex. Thus, the labeling agent can be a labeled polypeptide or a labeled antibody that specifically recognizes the already bound target polypeptide. Alternatively, the labeling agent can be a third moiety, such as another antibody, that specifically binds to the capture agent/polypeptide complex.

Other proteins capable of specifically binding immunoglobulin constant regions, such as protein A or protein G can also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

Preferred immunoassays for detecting the target polypeptide(s) are either competitive or noncompetitive. Noncompetitive immunoassays are assays in which the amount of captured analyte is directly measured. In one preferred “sandwich” assay, for example, the capture agents (antibodies) can be bound directly to a solid substrate where they are immobilized. These immobilized antibodies then capture the target polypeptide present in the test sample. The target polypeptide thus immobilized is then bound by a labeling agent, such as a second antibody bearing a label.

In competitive assays, the amount of analyte (NRRG or reporter gene encoded polypeptide) present in the sample is measured indirectly by measuring the amount of an added (exogenous) analyte displaced (or competed away) from a capture agent (antibody) by the analyte present in the sample. In one competitive assay, a known amount of, in this case, labeled polypeptide is added to the sample and the sample is then contacted with a capture agent. The amount of labeled polypeptide bound to the antibody is inversely proportional to the concentration of target polypeptide present in the sample.

In one embodiment, the antibody is immobilized on a solid substrate. The amount of target polypeptide bound to the antibody can be determined either by measuring the amount of target polypeptide present in a polypeptide/antibody complex, or alternatively by measuring the amount of remaining uncomplexed polypeptide.

The immunoassay methods of the present invention include an enzyme immunoassay (EIA) which utilizes, depending on the particular protocol employed, unlabeled or labeled (e.g., enzyme-labeled) derivatives of polyclonal or monoclonal antibodies or antibody fragments or single-chain antibodies that bind NRRG encoded polypeptide(s), either alone or in combination. In the case where the antibody that binds the target polypeptide(s) is not labeled, a different detectable marker, for example, an enzyme-labeled antibody capable of binding to the monoclonal antibody which binds the target polypeptide, can be employed. Any of the known modifications of EIA, for example, enzyme-linked immunoabsorbent assay (ELISA), can also be employed. As indicated above, also contemplated by the invention are immunoblotting immunoassay techniques such as western blotting employing an enzymatic detection system.

The immunoassay methods of the invention can also be other known immunoassay methods, for example, fluorescent immunoassays using antibody conjugates or antigen conjugates of fluorescent substances such as fluorescein or rhodamine, latex agglutination with antibody-coated or antigen-coated latex particles, haemagglutination with antibody-coated or antigen-coated red blood corpuscles, and immunoassays employing an avidin-biotin or streptavidin-biotin detection systems, and the like.

The particular parameters employed in the immunoassays of the present invention can vary widely, depending on various factors such as the concentration of antigen in the sample, the nature of the sample, the type of immunoassay employed and the like. Optimal conditions can be readily established by those of ordinary skill in the art. In certain embodiments, the amount of antibody that binds NRRG encoded polypeptide(s) is typically selected to give 50% binding of detectable marker in the absence of sample. If purified antibody is used as the antibody source, the amount of antibody used per assay will generally range from about 1 ng to about 100 ng. Typical assay conditions include a temperature range of about 4° C. to about 45° C., preferably about 25° C. to about 37° C., and most preferably about 25° C., a pH value range of about 5 to 9, preferably about 7, and an ionic strength varying from that of distilled water to that of about 0.2M sodium chloride, preferably about that of 0.15M sodium chloride. Times will vary widely depending upon the nature of the assay, and generally range from about 0.1 minute to about 24 hours. A wide variety of buffers, for example PBS, can be employed, and other reagents such as salt to enhance ionic strength, proteins such as serum albumins, stabilizers, biocides and non-ionic detergents can also be included.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

Antibodies for use in the various immunoassays described herein are commercially available or can be produced as described below.

Antibodies to NRRG or Reporter Gene Encoded Polypeptides, Nuclear Receptor Modulator Complexes, and/or DHRS.

Either polyclonal or monoclonal antibodies can be used in the immunoassays of the invention described herein, e.g., for the detection of NRRG or reporter gene encoded polypeptides, for the detection of the nuclear receptor modulator complexes, for the detection of a DHRS and the like. The techniques used to develop polyclonal antibodies are known in the art (see, e.g., Methods of Enzymology, “Production of Antisera With Small Doses of Immunogen: Multiple Intradermal Injections”, Langone, et al. eds. (Acad. Press, 1981)). Polyclonal antibodies produced by the animals can be further purified, for example, by binding to and elution from a matrix to which the peptide to which the antibodies were raised is bound. Those of skill in the art will know of various techniques common in the immunology arts for purification and/or concentration of polyclonal antibodies, as well as monoclonal antibodies see, for example, Coligan, et al. (1991) Unit 9, Current Protocols in Immunology, Wiley Interscience); Paul, (1999), Fundamental Immunology, 4^{th} Edition, Lippincott Williams & Wilkins Publishers, and references cited within.

Antibodies produced can also be monoclonal antibodies (“mAb's”). The term “antibody” as used in this invention includes intact molecules as well as fragments thereof, such as, Fab and F(ab′)₂′, and/or single-chain antibodies (e.g. scFv) which are capable of binding an epitopic determinant. Also, in this context, the term “mab's of the invention” refers, e.g., to monoclonal antibodies with specificity for a NRRG encoded polypeptide or a DHRS or nuclear receptor modulator complex. The general method used for production of hybridomas secreting mAbs is well known (Kohler and Milstein (1975) Nature, 256:495).

Antibodies fragments, e.g. single chain antibodies (scFv or others), can also be produced/selected using phage display technology. See, e.g., McCafferty et al. (1990) Nature, 348: 552-554; and, Hoogenboom et al. (1991) Nucleic Acids Res. 19: 4133-4137

Human antibodies can be produced without prior immunization by displaying very large and diverse V-gene repertoires on phage. See, e.g., Marks et al. (1991) J. Mol. Biol. 222: 581-597. In one embodiment natural VH and VL repertoires present in human peripheral blood lymphocytes are were isolated from unimmunized donors by PCR. The V-gene repertoires were spliced together at random using PCR to create a scFv gene repertoire which is was cloned into a phage vector to create a library of 30 million phage antibodies (Id.). From this single “naive” phage antibody library, binding antibody fragments have been isolated against more than 17 different antigens, including haptens, polysaccharides and proteins. See, e.g., Marks et al. (1991) J. Mol. Biol. 222: 581-597; Marks et al. (1993). Bio/Technology. 10: 779-783; Griffiths et al. (1993) EMBO J. 12: 725-734; and, Clackson et al. (1991) Nature. 352: 624-628. Antibodies have been produced against self proteins, including human thyroglobulin, immunoglobulin, tumor necrosis factor and CEA (Griffiths et al. (1993) EMBO J. 12: 725-734). It is also possible to isolate antibodies against cell surface antigens by selecting directly on intact cells. The antibody fragments are highly specific for the antigen used for selection and have affinities in the 1 nM to 100 nM range (Marks et al. (1991) J. Mol. Biol. 222: 581-597; Griffiths et al. (1993) EMBO J. 12: 725-734). Larger phage antibody libraries result in the isolation of more antibodies of higher binding affinity to a greater proportion of antigens.

It will also be recognized that antibodies can be prepared by any of a number of commercial services (e.g., Bethyl Laboratories (Montgomery, Tex.), Anawa (Switzerland), Eurogentec (Belgium and in the US in Philadelphia, Pa., etc.).

Scoring the Assay(s).

The assays of this invention are scored according to standard methods well known to those of skill in the art. The assays of this invention are typically scored as positive where there is a difference between, e.g., the level of dimer/heterodimer formation, the level or number of cofactor interactions, or activity seen, with the test agent present or with a greater amount of the test agent present or where the test agent has been previously applied, and the (usually negative) control. In certain preferred embodiments, the change/difference is a statistically significant change/difference, e.g. as determined using any statistical test suited for the data set provided (e.g. t-test, analysis of variance (ANOVA), semiparametric techniques, non-parametric techniques (e.g. Wilcoxon Mann-Whitney Test, Wilcoxon Signed Ranks Test, Sign Test, Kruskal-Wallis Test, etc.). Preferably the difference/change is statistically significant at a greater than 80%, preferably greater than about 90%, more preferably greater than about 98%, and most preferably greater than about 99% confidence level. Most preferred “positive” assays show at least a 1.2 fold, preferably at least a 1.5 fold, more preferably at least a 2 fold, and most preferably at least a 4 fold or even a 10-fold difference from the negative control.

High Throughput Screening

Any of the assays for compounds modulating dimer/heterodimer formation, modulating nuclear receptor and cofactor molecules and/or the inactivation of a nuclear receptor described herein are amenable to high throughput screening. For example, assays include detecting increases or decreases in NRRG or reporter gene transcription and/or translation in response to the presence of a test compound.

The cells utilized in the methods of this invention need not be contacted with a single test agent at a time. To the contrary, to facilitate high-throughput screening, a single cell can be contacted by at least two, preferably by at least 5, more preferably by at least 10, and most preferably by at least 20 test compounds. If the cell scores positive, it can be subsequently tested with a subset of the test agents until the agents having the activity are identified.

High throughput assays for various reporter gene products are well known to those of skill in the art. For example, multi-well fluorimeters are commercially available (e.g., from Perkin-Elmer). In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols of the various high throughputs. Thus, for example, Zymark Corp. provides technical bulletins describing screening systems for detecting the modulation of gene transcription, ligand binding, and the like.

Further Refinement

After such confirmation or testing, the agents of the invention can be further refined by generating full or partial nuclear receptor protein crystals with an agent of the invention bound to the DHRS of the nuclear receptor. The structure of the agent can then be further refined using chemical modification methods for three dimensional models to improve activity or affinity of the agent and to make second generation agents with improved properties.

Libraries of the Invention

The present invention provides a variety of libraries, including libraries of modulators and receptor/modulator complexes. For example, in one aspect, the invention provides libraries of modulators for a nuclear receptor, in which the library comprises a plurality of different modulators. More than one of the different modulators specifically binds a nuclear receptor dimer/heterodimer regulatory site (DHRS) of a nuclear receptor. The plurality of members present in the libraries of the present invention can range from a few members (e.g., about 5 or 10 members) to populations having about 50, 100, 500, 1000 or more members.

Not all of the modulators in the library necessarily need to bind the DHRS, i.e., mixed libraries comprising compounds the can or cannot bind the DHRS can be made and screened in the assays of the invention. The precise percentage can be selected by the user based, e.g., upon the intended use for the library.

Similarly, the library of modulators is optionally formatted in an arrangement of elements that comprises non-modulators (unrelated molecules, native ligands, or the like). The library of modulators is made up of the modulator members of the arrangement of elements, rather than the non-modulator elements. The overall arrangement of modulators and non-modulators can be referred to as a mixed element library.

The precise physical layout of the library is at the discretion of the practitioner. One can conveniently utilize gridded arrays of library members, e.g., formatted in a microtiter dish, or dried on a substrate such as a membrane, but other arrangements, are entirely appropriate, including those in which the library members are stored in separate locations that are accessed by one or more access control elements (e.g., that comprise a database of library member locations). The library format can be accessible by conventional robotics, or microfluidic devices, or a combination thereof.

One common array format for use is a microtiter plate array, in which the library comprises an array embodied in the wells of a microtiter tray (or the components therein). Such trays are commercially available and can be ordered in a variety of well sizes and numbers of wells per tray, as well as with any of a variety of functionalized surfaces for binding of assay or array components. Common trays include the ubiquitous 96 well plate, with 384 and 1536 well plates also in common use.

In addition to libraries that comprise liquid phase arrays, modulator components can be stored in libraries comprising solid phase arrays of modulators. These arrays fix materials in a spatially accessible pattern (e.g., a grid of rows and columns) onto a solid substrate such as a membrane (e.g., nylon or nitrocellulose), a polymer or ceramic surface, a glass or modified silica surface, a metal surface, or the like. Components can be accessed, e.g., by local rehydration (e.g., using a pipette or other fluid handling element) and fluidic transfer, or by scraping the array or cutting out sites of interest from the array.

While component libraries are most often thought of as physical elements with a specified spatial-physical relationship, the present invention can also make use of “logical” libraries, which do not have a straightforward spatial organization. For example, a computer system can be used to track the location of one or several components of interest, which are located in or on physically disparate components. The computer system creates a logical library by providing a “look-up” table of the physical location of array members (e.g., using a commercially available inventory tracking system). Thus, even components in motion can be part of a logical library, as long as the members of the library can be specified and located.

The libraries of the invention optionally include any of the physical components of the invention described anywhere herein, including modulators (including modulators having any physical structure noted herein), modulator/receptor complexes (including those having any physical structure noted herein), DHRSs or the like. The receptor can be any of those noted herein, e.g., TR, GR, ER, etc. In preferred embodiments, members of the modulator library include a plurality of different modulators that specifically bind a nuclear receptor dimer/heterodimer regulatory site (DHRS) of a nuclear receptor.

Indeed, virtually any test agent can be formatted into a library and screened as a putative modulator according to the methods of this invention. Such agents include, but are not limited to, small organic molecules, nucleic acids, proteins (e.g., polypeptide, antibody, or fragment thereof), peptides, sugars, polysaccharides, glycoproteins, lipids, and the like. The term “small organic molecules” typically refers to molecules of a size comparable to those organic molecules generally used as pharmaceuticals. The term excludes biological macromolecules (e.g., proteins, nucleic acids, etc.). In certain embodiments, a peptide is, e.g., less than 15 amino acids, less than 10 amino acids, less than 8 amino acids, etc. In certain embodiments, the peptide is unrestrained, while in other embodiments, the peptide can be cyclized or constrained. The peptide can be composed of natural, synthetic or a combination of natural and synthetic amino acids. In certain embodiments, the test agent is not an antibody, or is not a protein or is not a nucleic acid.

Conventionally, new chemical entities with useful properties are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one preferred embodiment, high throughput screening methods involve providing a library containing a large number of potential therapeutic compounds (candidate compounds). Such “combinatorial chemical libraries” are then screened in one or more assays, as described herein to identify those library members (particular chemical species or subclasses) that display a desired characteristic modulator activity. The compounds thus identified can serve as conventional “lead compound” or can themselves be used as modulators, including as potential or actual therapeutics.

A combinatorial chemical library is a collection of diverse compounds generated by chemical synthesis, or biological synthesis (or both), by combining a number of chemical “building blocks” such as reagents. For example, a linear combinatorial -chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) J. Med. Chem. 37:1233-1250).

Preparation of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res. 37: 487-493, Houghton et al. (1991) Nature, 354: 84-88); peptoids (PCT Publication No WO 91/19735, 26 December 1991); encoded peptides (PCT Publication WO 93/20242, 14 October 1993); phage display libraries (see, e.g., Smith and Petrenko, (1997), “Phage Display”, Chem. Rev., 97:391-410; and, Geistlinger and Guy “An inhibitor of the interaction of thyroid hormone receptor beta and glucocorticoid interacting protein 1” J. Am. Chem. Soc. 2001 Feb. 21;123(7):1525-6); random bio-oligomers (PCT Publication WO 92/00091, 9 January 1992); benzodiazepines (U.S. Pat. No. 5,288,514); diversomers such as hydantoins, benzodiazepines and dipeptides (Hobbs et al., (1993) Proc. Nat. Acad. Sci. USA 90: 6909-6913); vinylogous polypeptides (Hagihara et al. (1992) J. Amer. Chem. Soc. 114: 6568); nonpeptidal peptidomimetics with a Beta-D-Glucose scaffolding (Hirschmann et al., (1992) J. Amer. Chem. Soc. 114: 9217-9218); analogous organic syntheses of small compound libraries (Chen et al. (1994) J. Amer. Chem. Soc. 116: 2661); oligocarbamates (Cho, et al., (1993) Science 261:1303), peptidyl phosphonates (Campbell et al., (1994) J. Org. Chem. 59: 658); Gordon et al., (1994) J. Med. Chem. 37:1385, nucleic acid libraries (see, e.g., Strategene, Corp.); peptide nucleic acid libraries (see, e.g., U.S. Pat. No. 5,539,083); antibody libraries (see, e.g., Vaughn et al. (1996) Nature Biotechnology, 14(3): 309-314), and PCT/US96/10287); carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. No. 5,525,735 and 5,519,134, morpholino compounds U.S. Pat. No. 5,506,337, benzodiazepines U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, in certain embodiments, commercially available libraries can be accessed for test agents or agents.

A number of well-known robotic systems have also been developed for solution phase chemistries, which can be used for combinatorial synthesis. These systems include, but are not limited to, automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic manual synthetic operations performed by a chemist, and the Venture™ platform, an ultra-high-throughput synthesizer that can run between 576 and 9,600 simultaneous reactions from start to finish (see, Advanced ChemTech, Inc. Louisville, Ky.)). Microfluidic approaches can also be used for library generation and screening, e.g., using a microfluidic device comprising an interface that can access standard microtiter plates, or that can access arrays of dried reagents such as the LibraryCard™ from Caliper Technologies, Corp. (Mountain View, Calif.). Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J.; Asinex, Moscow, Russia; Tripos, Inc., St. Louis, Mo.; ChemStar Ltd., Moscow, Russia; 3D Pharmaceuticals, Exton, Pa.; Martek Biosciences, Columbia, Md., etc.).

Agent Databases

In certain embodiments, agents that score positively in the assays described herein (e.g., show an ability to modulate, e.g., dimer/heterodimer formation, nuclear receptor-cofactor molecule interactions, nuclear receptor activation, nuclear receptor-dependent gene expression, etc.) can be entered into a database of putative and/or actual agents (modulators). The term database refers to a system for recording and retrieving information (e.g., a computer comprising database software, or a manual database). In preferred embodiments, the database also provides means for sorting and/or searching the stored information (e.g., appropriate software or an appropriate index). The database can comprise any convenient media including, but not limited to, paper systems, card systems, mechanical systems, electronic systems, optical systems, magnetic systems or combinations thereof. Preferred databases include electronic (e.g., computer-based) databases. Computer systems for use in storage and manipulation of databases are well known to those of skill in the art and include, but are not limited to personal computer systems, mainframe systems, distributed nodes on an inter- or intra-net, data or databases stored in specialized hardware (e.g., in microchips), and the like. As mentioned above, the database can include an inventory tracking/storage/control system that tracks modulators, complexes, libraries, library members, or mixed library members, as described herein.

Treatment and Pharmaceutical Compositions

A wide variety of disease conditions are treatable with appropriate nuclear receptor agents (modulators). These include, but are not limited to, modulation of reproductive organ function, hyperthyroidism, aldosteronism, Cushing's syndrome, hirsutism, hypercholesterolemia, hyperlipidermia, atherosclerosis, obesity, cardiac arrhythmia, hypothyroidism, osteoporosis, hypertension, glaucoma, depression, inflammation, immunomodulation, diabetes, depression and/or cancer (e.g., bone cancer, ovarian cancer, thyroid cancer, breast cancer, prostate cancer, etc.), etc.

In general, a therapeutically effective amount of the modulator is administered over time. In therapeutic use, the compounds of the present invention are usually administered in a standard pharmaceutical formulation. The present invention therefore provides pharmaceutical compositions comprising a modulator of the invention (or deliverable form thereof, such as a pharmaceutically acceptable salt). In certain embodiments, the agent (modulator) is mixed with one or more pharmaceutically acceptable excipients or carriers prior to administration. Pharmaceutical administration methods include those that bring the composition into contact with a target tissue or fluid, e.g., via oral, intravenous, parenteral, topical (including ocular), or rectal administration.

Agents (modulators) of the invention can also be used for combination therapy. In certain embodiments, an agent of the invention is co-administered with an agonist or an antagonist a nuclear receptor. In one aspect of the invention, the co-administration of the agent and the agonist or the antagonist of the nuclear receptor counteracts at least one deleterious effect of the agonist or the antagonist. For example, steroids with glucocorticoid activity are used extensively as immunosuppressant and anti-inflammatory agents. However, the benefits of this therapy are countered by deleterious effects of the steroids. Many of the beneficial effects do not require dimerization of the glucocorticoid receptor to be elicited, while many of the deleterious effects appear to require receptor dimer formation. For example, glucocorticoids have an undesirable effect, e.g., increasing blood sugar, acting through the first mechanism of action of a nuclear receptor, described herein, while glucocorticoids have desirable anti-inflammatory effects when the nuclear receptor is acting through the second mechanism of action described herein. Thus, an agent of the invention that modulates dimer/heterodimer formation can be administered with a nuclear agonist, e.g., a steroid with glucocorticoid activity, to selectively modulate the receptor's, e.g., the glucocorticoid receptor's, actions.

Many responses to other nuclear receptors display the same pattern. Some nuclear receptors require homodimerization or heterodimerization on DNA, whereas others do not require this. This pattern is true for the estrogen receptor. In certain embodiments, agents of the invention can be used in conjunction with, e.g., an estrogen or selective estrogen, receptor modulator (SERM) to elicit even more selective, e.g., estrogen, receptor responses. Others nuclear receptors can work as dimers or monomers. For example, the thyroid receptor can bind DNA and activate gene transcription either as homodimers, heterodimers with retinoid X receptor or as monomers. Agents of the invention can be designed to discriminate thyroid receptor action at these response elements.

In general, pharmaceutically useful substances identified by the methods of this invention can be useful in the form of the free acid, in the form of a salt and/or as a hydrate. All forms are within the scope of the invention. Basic salts can be formed and are a convenient form for use; in practice, use of the salt form inherently amounts to use of the acid form. The bases which can be used to prepare the salts include preferably those which produce, when combined with the free acid, pharmaceutically acceptable salts, that is, salts whose anions are non-toxic to the animal organism in pharmaceutical doses of the salts, so -that the beneficial properties inherent in the free acid are not vitiated by side effects ascribable to the cations. Although pharmaceutically acceptable salts of the acid compound are preferred, all salts are useful as sources of the free acid form even if the particular salt per se is desired only as an intermediate product as, for example, when the salt is formed only for purposes of purification and identification, or when it is used as an intermediate in preparing a pharmaceutically acceptable salt by ion exchange procedures.

In any case, the modulators of the invention can be administered to a mammalian host in a variety of formats, e.g., they can be combined with various pharmaceutically acceptable inert carriers in the form of tablets, capsules, lozenges, troches, hard candies, powders, sprays, elixirs, syrups, injectable or eye drop solutions (e.g., for treatment of glaucoma), or in ocular implants or contact lenses and/or the like depending on the chosen route of administration, e.g., orally, topically, or parenterally. Parenteral administration in this respect includes administration by the following routes: intravenous, intramuscular, subcutaneous, intraocular, intrasynovial, transepithelial (including transdermal, ophthalmic, sublingual and buccal), topical (including ophthalmic, dermal, ocular, rectal, nasal inhalation via insufflation and aerosol), and rectal systemic. Oral administration is one preferred route of administration.

Active compounds can be orally administered, for example, with an inert diluent or with an assimilable edible carrier, or it can be enclosed in hard or soft shell gelatin capsules, or it can be compressed into tablets, or it can be incorporated directly with food in the diet. For oral therapeutic administration, the active compound can be incorporated with excipient and used in the form of ingestible tablets, buccal tablets, troches, capsules, elixirs, suspensions, syrups, wafers, and the like. Such compositions and preparations should contain at least 0.1% of active compound (modulator). The percentage of the compositions and preparations can, of course, be varied and can conveniently be, e.g., between about 2 and about 20% of the weight of the unit. The amount of active compound in such therapeutically useful compositions is such that a suitable dosage will be obtained. Preferred compositions or preparations according to the present invention are prepared so that an oral dosage unit form contains between about 0.05 and 1000 mg of active compound.

One advantage of a tablet or a capsule is that the patient can easily self-administer unit doses. In general, unit doses contain, e.g., in the range of from 0.05-100 mg of a given modulator. The active ingredient can be administered, e.g., from 1 to about 10 times a day. Thus, daily doses are in general in the range of from 0.05 to 1000 mg per day.

The tablets, troches, pills, capsules and/or the like can also contain the following: a binder such as polyvinylpyrrolidone, gum tragacanth, acacia, sucrose, corn starch or gelatin; an excipient such as calcium phosphate, sodium citrate and calcium carbonate; a disintegrating agent such as corn starch, potato starch, tapioca starch, certain complex silicates, alginic acid and the like; a lubricant such as sodium lauryl sulfate, talc and magnesium stearate; a sweetening agent such as sucrose, lactose or saccharin; or a flavoring agent such as peppermint, oil of wintergreen or cherry flavoring. Solid compositions of a similar type are also employed as fillers in soft and hard-filled gelatin capsules; preferred materials in this connection also include lactose or milk sugar as well as high molecular weight polyethylene glycols. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the active compound, sucrose as a sweetening agent, methyl and propylparabens as preservatives, a dye, flavoring such as cherry or orange flavor, emulsifying agents and/or suspending agents, as well as such diluents as water, ethanol, propylene glycol, glycerin and various like combinations thereof. Of course, any material used in preparing any dosage unit form should be pharmaceutically pure and substantially non-toxic in the amounts employed. In addition, the active compound can be incorporated into sustained-release preparations and formulations.

The active compound can also be administered parenterally or intraperitoneally. For purposes of parenteral administration, solutions in sesame or peanut oil or in aqueous propylene glycol can be employed, as well as sterile aqueous solutions of the corresponding water-soluble, alkali metal or alkaline-earth metal salts. Such aqueous solutions should be suitably buffered, if necessary, and the liquid diluent first rendered isotonic with sufficient saline or glucose. Solutions of the active compound as a free base or a pharmacologically acceptable salt can be prepared in water suitably mixed with a surfactant such as hydroxypropylcellulose. A dispersion can also be prepared in glycerol, liquid polyethylene glycols and mixtures thereof and in oils. Under ordinary conditions of storage and use, these preparations contain a preservative to prevent the growth of microorganisms. These particular aqueous solutions are especially suitable for intravenous, intramuscular, subcutaneous and intraperitoneal injection purposes. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifingal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating the active compound in the required amount in the appropriate solvent with various of the other ingredients enumerated above, as required, followed by filtered sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from the previously sterile-filtered solution thereof.

For purposes of topical administration, dilute sterile, aqueous solutions (usually in about 0.1% to 5% concentration, though this can vary depending on the solubility of the modulator, the desired dose and the like), otherwise similar to the above parenteral solutions, are prepared in containers suitable for drop-wise administration to the eye. The therapeutic compounds of this invention can be administered to a mammal alone or in combination with pharmaceutically acceptable carriers. As noted above, the relative proportions of active ingredient and carrier are determined by the solubility and chemical nature of the compound, chosen route of administration and standard pharmaceutical practice. The dosage of the modulators that are most suitable for prophylaxis or treatment will vary with the form of administration, the particular compound chosen and the physiological characteristics of the particular patient under treatment. Generally, small dosages will be used initially and, if necessary, will be increased by small increments until the optimum effect under the circumstances is reached. Oral administration generally uses higher dosages. The compounds are administered either orally or parenterally, or topically as eye drops or via an ocular insert (e.g., an modulator impregnated contact lens). Dosages can readily be determined by physicians using methods known in the art, using dosages typically determined from animal studies or available modulator therapies as starting points.

Where the modulator is used in combination with another therapeutic agent, the effective amount of the modulator can, in some circumstances, be lower than the effective amount of modulator administered without the additional therapeutic. The delivery method can also vary depending on what is co-administered with the modulator.

In general, the typical daily dose of modulator of the invention varies according to individual needs, the condition to be treated and with the route of administration. Suitable doses are typically in the general range of from 0.001 to 10 mg/kg bodyweight of the recipient per day. Within this general dosage range, doses can be chosen at which the modulators have desired effects, e.g., which lower plasma cholesterol levels and raise metabolic rate with little or no direct effect on the heart. In general, such doses will be in the range of from lower doses (0.001 to 0.5 mg/kg) to higher doses (0.5 to 10 mg/kg). Similarly, within the general dose range, doses can be chosen at which the modulators lower plasma cholesterol levels and have little or no effect on the heart without raising metabolic rate. In general, but not exclusively, such doses will be in the range of from 0.001 to 0.5 mg/kg. It is to be understood that the sub ranges noted above are not mutually exclusive and that the particular activity encountered at a particular dose will depend on the nature of the modulator used.

Receptor Cloning and Assay Tissue Culture

In practicing the present invention, many conventional techniques in molecular biology, microbiology, and recombinant DNA are advantageously used. For example, receptors are optionally cloned and expressed, e.g., to perform in vitro or in vivo assay screens as described above. In general, these techniques are well known and are explained in, for example, Current Protocols in Molecular Biology, Volumes I, II, and III, 1997 (F. M. Ausubel ed.), supplemented through 2002; Sambrook et al., 2001, Molecular Cloning: A Laboratory Manual, Third Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series. Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

Similarly, cells (e.g., mammalian, fungal, plant or animal cells) comprising receptors can be grown, e.g., using conventional culture methods. In addition to the references noted in the preceding paragraph, further details regarding tissue culture can be found, e.g., in Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (Eds.) (1995) Plant Cell, Tissue and Organ Culture; Fundamental Methods Springer Lab Manual, Springer-Verlag (Berlin Heidelberg New York) and Atlas and Parks (Eds.) The Handbook of Microbiological Media (1993) CRC Press, Boca Raton, Fla.

Receptors are optionally purified for in vitro or in vivo use, e.g., for producing the receptor-modulator complexes of the invention. In addition to other references noted herein, a variety of purification/protein purification methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2nd Edition Wiley-Liss, NY; Walker (1996) The Protein Protocols Handbook Humana Press, NJ; Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3rd Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and the references cited therein.

Systems

Systems are also features of the invention. In one embodiment, a system includes a screening system for screening test agents that modulate dimer/heterodimer formation and/or cofactor molecule interactions of nuclear receptors. For example, the screening system includes at least one polypeptide (e.g., a full or partial nuclear receptor amino acid sequence), where the at least one polypeptide comprises a nuclear receptor dimer/heterodimer regulatory site (DHRS); and, instructions for detecting dimer/heterodimerization and/or interactions of cofactor molecules of the at least one polypeptide. In certain embodiments, the polypeptide is provided by a nucleic acid, which encodes the polypeptide. A prescreening system for prescreening a test agent that binds to a nuclear receptor dimer/heterodimer regulator site (DHRS) is also provided. The prescreening system includes a polypeptide that comprises the DHRS; and, instructions for detecting specific binding of the test agent to the DHRS.

The invention also provides for a system for designing putative compounds that contact a nuclear receptor dimer/heterodimer regulatory site (DHRS). For example, the system includes a three dimensional model of a protein or polypeptide comprising a nuclear receptor dimer/heterodimer regulatory site (DHRS). The system also typically includes features for user-interface with the model and, e.g., instructions for modeling binding of one or more compounds to the three dimensional model to design at least one putative compound that contacts the DHRS.

The systems of the invention optionally include computers, databases, etc. For example, see the section on Agent Databases above.

Kits

Another aspect of the invention is to provide kits for carrying out the subject methods. For example, kits can include the receptor complexes of the invention, in combination with other kit components, such as packaging materials, instructions for user of the complexes or the like. Libraries can also be packaged in kits, e.g., comprising library components such as arrays in combination with packaging materials, instructions for array use or the like. Kits generally contain one or more reagents necessary or useful for practicing the methods of the invention. Reagents can be supplied in pre-measured units so as to provide for uniformity and precision in test results.

It is understood that the examples and embodiments described herein are for illustrative purposes only and that various modifications or changes in light thereof will be suggested to persons skilled in the art and are to be included within the spirit and purview of this application and scope of the appended claims.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method of screening for a test agent that modulates dimer/heterodimer formation or cofactor interaction with a nuclear receptor, the method comprising:
contacting at least one nuclear receptor dimer/heterodimer regulatory site (DHRS) of at least one nuclear receptor with a test agent; and,
detecting a change in a level of dimer/heterodimer formation or a change in cofactor interaction with the at least one nuclear receptor that is mediated by the test agent, thereby screening for the test agent that modulates dimer/heterodimer formation or cofactor interaction with the nuclear receptor.

2. The method of claim 1, wherein the at least one DHRS comprises a hydrophobic cluster, and wherein the hydrophobic cluster is located on a surface of the nuclear receptor.

3. The method of claim 2, wherein the at least one DHRS further comprises a region comprising polar and non-polar amino acids proximal to the hydrophobic cluster.

4. The method of claim 3, wherein the at least one DHRS further comprises a solvent-based region.

5. The method of claim 1, wherein the at least one DHRS comprises residues Valine 376, Leucine 400, Leucine 422, and Valine 425 of a thyroid hormone receptor P.

6. The method of claim 5, wherein the at least one DHRS further comprises residues Serine 381, Aspartate 382, Glutamate 393, Glutamate 396, and Arginine 429 of the thyroid hormone receptor β.

7. The method of claim 6, wherein the at least one DHRS further comprises a solvent-based region.

8. The method of claim 1, wherein the at least one DHRS comprises residues Valine 322, Leucine 346, Leucine 368, and Valine 371 of a thyroid hormone receptor α.

9. The method of claim 1, wherein the at least one DHRS comprises residues Alanine 381, Valine 405, Leucine 427, and Methionine 430 of a peroxisome proliferator activated α receptor.

10. The method of claim 1, wherein the at least one DHRS comprises residues Valine 390, Leucine 414, Leucine 436, and Methionine 439 of a peroxisome proliferator activated γ receptor.

11. The method of claim 1, wherein the at least one DHRS comprises residues Isoleucine 332, Leucine 356, Leucine 378, and Isoleucine 381 of a retinoic acid α receptor.

12. The method of claim 1, wherein the at least one DHRS comprises residues Isoleucine 346, Alanine 370, Methionine 394, and Leucine 397 of a pregnane X receptor.

13. The method of claim 1, wherein the at least one DHRS comprises residues Isoleucine 336, Serine 360, Isoleucine 384, and Leucine 387 of a vitamin D receptor.

14. The method of claim 1, wherein the at least one DHRS comprises residues Leucine 810, Isoleucine 835, Threonine 860, and Leucine 863 of an androgen receptor.

15. The method of claim 1, wherein the at least one DHRS comprises residues Isoleucine 451, Threonine 483, Leucine 508, and Leucine 511 of an estrogen receptor.

16. The method of claim 1, wherein the at least one DHRS comprises residues Leucine 824, Isoleucine 849, Threonine 874, and Leucine 877 of a progesterone receptor.

17. The method of claim 1, wherein the agent masks residues in the at least one DHRS of the at least one nuclear receptor, thereby preventing dimer/heterodimer formation.

18. The method of claim 1, wherein the at least one nuclear receptor is selected from the group consisting of: a thyroid hormone receptor, a glucocorticoid receptor, an estrogen receptor, an androgen receptor, a mineralocorticoid receptor, a progestin receptor, a vitamin D receptor, a retinoid receptor, a retinoid X receptor, a peroxisomal proliferator activated receptor, an estrogen-receptor related receptor, a short heterodimer partner, a constitutive androstane receptor, a liver X receptor, a pregnane X receptor, a HNF-4 receptor, a farnesoid X receptor, and an orphan receptor.

19. The method of claim 1, further comprising:
comparing a change in a level of dimer/heterodimer formation of the at least one nuclear receptor to a level of dimer/heterodimer formation in a control, wherein a difference in the level of dimer/heterodimer formation in the contacted DHRS and the level in the control indicates that the agent alters dimer/heterodimer formation of the at least one nuclear receptor.

20. The method of claim 19, wherein the control is exposed to a lower concentration of the test agent.

21. The method of claim 20, wherein the lower concentration is the absence of said test agent.

22. The method of claim 1, wherein the agent modulates an interaction of the at least one nuclear receptor and a cofactor molecule.

23. The method of claim 1, wherein the change in the level of dimer/heterodimer formation or the change in cofactor molecule interaction correlates with an activation of the at least one nuclear receptor.

24. The method of claim 1, wherein the change in the level of dimer/heterodimer formation or the change in cofactor molecule interaction correlates with a repression of the at least one nuclear receptor activity.

25. The method of claim 1, wherein the level of dimer/heterodimer formation or thc change in cofactor molecule interaction is detected by detecting expression of at least one nuclear receptor responsive gene or reporter gene.

26. The method of claim 1, wherein the level of dimer/heterodimer formation or the change in cofactor molecule interaction is detected by detecting nuclear receptor activation.

27. The method of claim 1, wherein the level of dimer/heterodimer formation or the change in cofactor molecule interaction is detected by detecting nuclear receptor repression.

28. The method of claim 1, wherein the level of dimer/heterodimer formation or the change in cofactor molecule interaction is detected by a gel shift assay, a fluorescence assay, a chromatography assay, an immunochemistry assay, a fusion tag assay, or a two hybrid assay.

29. The method of claim 1, wherein the at least one nuclear receptor comprises at least two nuclear receptors.

30. The method of claim 29, wherein one of the at least two nuclear receptors is a retinoid X receptor (RXR).

31. The method of claim 1, wherein the test agent is an agent other than an antibody.

32. The method of claim 1, wherein the test agent is an agent other than a protein.

33. The method of claim 1, wherein the test agent is a small organic molecule.

34. The method of claim 1, wherein the test agent is a peptide.

35. The method of claim 1, wherein the test agent is contacted directly to the at least one DHRS.

36. The method of claim 1, wherein the test agent is contacted to a cell containing the at least one DHRS.

37. The method of claim 1, wherein the test agent is contacted to an animal comprising a cell containing the at least one DHRS.

38. The method of claim 1, wherein detecting the change mediated by the test agent is performed in vitro.

39. The method of claim 1, wherein detecting the change mediated by the test agent is performed in vivo.

40. A nuclear receptor: agent complex produced by the method of claim 1.

41. The complex of claim 40, wherein the agent is GC-24.

42. The complex of claim 40, wherein the agent is an agent other than GC-24.

43. A method of treating a subject having a disease state which is alleviated by treatment with a nuclear receptor modulator, the method comprising administering a therapeutically effective amount of an agent of claim 1 to the subject in need thereof.

44. The method of claim 43, wherein the disease state is selected from the group consisting of: hyperthyroidism, aldosteronism, Cushing's syndrome, hirsutism, cancer, thyroid cancer, breast cancer, prostate cancer, bone cancer, ovarian cancer, hypercholesterolemia, hyperlipidemia, atherosclerosis, obesity, cardiac arrhythmia, modulation of reproductive organ function, hypothyroidism, osteoporosis, hypertension, glaucoma, and depression.

45. The method of claim 43, wherein the agent is mixed with one or more pharmaceutically acceptable excipients prior to said administering.

46. The method of claim 43, wherein the subject is a human.

47. The method of claim 43, wherein the subject is a non-human mammal.

48. The method of claim 43, wherein the agent is co-administered with an agonist or an antagonist of a nuclear receptor.

49. The method of claim 48, wherein the co-administration of the agent and the agonist or the antagonist of the nuclear receptor counteracts at least one deleterious effect of the agonist or the antagonist.

50. A method of prescreening for an agent that modulates dimer/heterodimer formation or cofactor molecule interaction of a nuclear receptor, the method comprising:
contacting a nuclear receptor dimer/heterodimer regulatory site (DHRS) with a test agent; and,
detecting a specific binding of the test agent to said DHRS.

51. The method of claim 50, wherein the specific binding indicates that the test agent is a candidate modulator of dimer/heterodimer formation or cofactor molecule interaction.

52. The method of claim 50, wherein the test agent is not an antibody.

53. The method of claim 50, wherein the test agent is not a protein.

54. The method of claim 50, wherein the test agent is a small organic molecule.

55. The method of claim 50, wherein the test agent is a peptide.

56. A method of designing a compound to contact a nuclear receptor dimer/heterodimer regulatory site (DHRS), the method comprising:
providing a three dimensional model of a protein or polypeptide comprising the DHRS; and,
modeling a binding of one or more compounds to the three dimensional model, thereby identifying one or more compound that binds to the DHRS.

57. The method of claim 56, wherein modeling of the binding comprises using a computer program to design a putative compound that binds to the DHRS.

58. The method of claim 57, wherein the computer program is selected from the group consisting of: DOCK, Catalyst and MCSS/Hook.

59. A nuclear receptor:bound compound complex produced by the method of claim 56.

60. The complex of claim 59, wherein the complex inhibits or reduces dimerization or heterodimerization of the nuclear receptor.

61. The complex of claim 59, wherein the complex inhibitors or reduces binding of one or more cofactor molecules to the nuclear receptor.

62. The complex of claim 59, wherein the complex inhibits an appropriate folding of the ligand binding domain of the nuclear receptor.

63. The complex of claim 59, wherein the complex inhibits activation of an AF-1 domain of the nuclear receptor.

64. A method of identifying one or more modulators for at least one nuclear receptor, the method comprising:
providing a plurality of putative modulators;
contacting at least one nuclear receptor dimer/heterodimer regulatory site (DHRS) of a nuclear receptor with the putative modulators, wherein at least one of the putative modulators binds the DHRS; and,
testing the putative modulators for modulator activity on the nuclear receptor, thereby identifying the one or more modulators of the nuclear receptor.

65. The method of claim 64, wherein the plurality of putative modulators comprises between 5 and 1000 members.

66. The method of claim 64, wherein the plurality of putative modulators comprises more than 1000 members.

67. The method of claim 64, wherein the nuclear receptor is selected from the group consisting of: a thyroid hormone receptor, a glucocorticoid receptor, an estrogen receptor, an androgen receptor, a mineralocorticoid receptor, a progestin receptor, a vitamin D receptor, a retinoid receptor, a retinoid X receptor, a peroxisomal proliferator activated receptor, an estrogen-receptor related receptor, a short heterodimer partner, a constitutive androstane receptor, a liver X receptor, a pregnane X receptor, a HNF-4 receptor, a farnesoid X receptor, and an orphan receptor.

68. The method of claim 64, wherein the testing comprises:
binding the plurality of putative modulators to the least one DHRS;
selecting members of the plurality of putative modulators that bind the at least one DHRS; and,
testing the resulting bound nuclear receptor for modulator activity.

69. The method of claim 64, wherein the modulator activity is nuclear receptor activation.

70. The method of claim 64, wherein the modulator activity is represssion of nuclear receptor activity.

71. The method of claim 64, wherein the modulator activity is a dimerization or heterodimerization activity.

72. The method of claim 64, wherein the testing is performed in vitro.

73. The method of claim 64, wherein the testing is performed in vivo.

74. A method of modulating nuclear receptor activation, the method comprising:
contacting a nuclear receptor dimer/heterodimer regulatory site (DHRS) of a nuclear receptor with an agent; wherein the agent preferentially binds the DHRS, thereby modulating nuclear receptor activation.

75. The method of claim 74, wherein the DHRS comprises a hydrophobic cluster and is located on the surface of the nuclear receptor.

76. The method of claim 75, wherein the DHRS further comprises a region comprising polar and non-polar amino acids proximal to the hydrophobic cluster.

77. The method of claim 76, wherein the DHRS further comprises a solvent-based region.

78. The method of claim 74, wherein the DHRS comprises residues corresponding to Valine 376, Leucine 400, Leucine 422 and Valine 425 of a thyroid hormone receptor β.

79. The method of claim 78, wherein the DHRS further comprises residues Serine 381, Aspartate 382, Glutamate 393, Glutamate 396, and Arginine 429 of the thyroid hormone receptor β.

80. The method of claim 79, wherein the DHRS further comprises a solvent-based region.

81. The method of claim 74, wherein the DHRS comprises residues Valine 322, Leucine 346, Leucine 368, and Valine 371 of a thyroid hormone receptor α.

82. The method of claim 74, wherein the DHRS comprises residues Alanine 381, Valine 405, Leucine 427 and Methionine 430 of a peroxisome proliferator activated α receptor.

83. The method of claim 74, wherein the DHRS comprises residues Valine 390, Leucine 414, Leucine 436 and Methionine 439 of a peroxisome proliferator activated γ receptor.

84. The method of claim 74, wherein the DHRS comprises residues Isoleucine 332, Leucine 356, Leucine 378 and Isoleucine 381 of a retinoic acid α receptor.

85. The method of claim 74, wherein the DHRS comprises residues Isoleucine 346, Alanine 370, Methionine 394 and Leucine 397 of a pregnane X receptor.

86. The method of claim 74, wherein the DHRS comprises residues Isoleucine 336, Serine 360, Isoleucine 384 and Leucine 387 of a vitamin D receptor.

87. The method of claim 74, wherein the at least one DHRS comprises residues Leucine 810, Isoleucine 835, Threonine 860, and Leucine 863 of an androgen receptor.

88. The method of claim 74, wherein the at least one DHRS comprises residues Isoleucine 451, Threonine 483, Leucine 508, and Leucine 511 of an estrogen receptor.

89. The method of claim 74, wherein the at least one DHRS comprises residues Leucine 824, Isoleucine 849, Threonine 874, and Leucine 877 of a progesterone receptor.

90. The method of claim 74, wherein the agent modulates activation of the nuclear receptor by inhibiting dimer or heterodimer formation of the nuclear receptor.

91. The method of claim 74, wherein the agent masks residues in the DHRS and prevents dimer/heterodimer formation, thereby modulating nuclear receptor activation.

92. The method of claim 74, wherein the agent modulates nuclear receptor activation by inhibiting activation of an AF-1 domain.

93. The method of claim 74, wherein the agent modulates nuclear receptor activation by activating an AF-1 domain.

94. The method of claim 74, wherein the agent modulates nuclear receptor activation by inhibiting activation of a liganded nuclear receptor.

95. The method of claim 74, wherein the agent modulates nuclear receptor activation by activating a liganded nuclear receptor.

96. The method of claim 74, wherein the agent modulates nuclear receptor activation by inhibiting activation of a unliganded nuclear receptor.

97. The method of claim 74, wherein the agent modulates nuclear receptor activation by activating a unliganded nuclear receptor.

98. The method of claim 74, wherein the agent modulates gene transcription.

99. The method of claim 74, wherein the nuclear receptor is selected from the group consisting of: a thyroid hormone receptor, a glucocorticoid receptor, an estrogen receptor, an androgen receptor, a mineralocorticoid receptor, a progestin receptor, a vitamin D receptor, a retinoid receptor, a retinoid X receptor, a peroxisomal proliferator activated receptor, an estrogen-receptor related receptor, a short heterodimer partner, a constitutive androstane receptor, a liver X receptor, a pregnane X receptor, a HNF-4 receptor, a farnesoid X receptor, and an orphan receptor.

100. The method of claim 74, wherein the nuclear receptor comprises a nuclear receptor isoform.

101. A nuclear receptor modulator complex comprising a nuclear receptor bound to an agent, wherein the agent preferentially binds a nuclear receptor dimer/heterodimer regulator site (DHRS) of the nuclear receptor.

102. The nuclear receptor modulator complex of claim 101, wherein the DHRS comprises a hydrophobic cluster and is located on the surface of the nuclear receptor.

103. The nuclear receptor modulator complex of claim 102, wherein the DHRS further comprises a region comprising polar and non-polar amino acids proximal to the hydrophobic cluster.

104. The nuclear receptor modulator complex of claim 103, wherein the DHRS further comprises solvent-accessible region.

105. The nuclear receptor modulator complex of claim 101, wherein the DHRS comprises residues corresponding to Valine 376, Leucine 400, Leucine 422 and Valine 425 of a thyroid hormone receptor β.

106. The nuclear receptor modulator complex of claim 105, wherein the DHRS further comprises residues Serine 381, Aspartate 382, Glutamate 393, Glutamate 396, and Arginine 429 of the thyroid hormone receptor β.

107. The nuclear receptor modulator complex of claim 106, wherein the DHRS further comprises a solvent-accessible region.

108. The nuclear receptor modulator complex of claim 101, wherein the DHRS comprises residues Valine 322, Leucine 346, Leucine 368, and Valine 371 of a thyroid hormone receptor α.

109. The nuclear receptor modulator complex of claim 101, wherein the DHRS comprises residues Alanine 381, Valine 405, Leucine 427 and Methionine 430 of a peroxisome proliferator activated α receptor.

110. The nuclear receptor modulator complex of claim 101, wherein the DHRS comprises residues Valine 390, Leucine 414, Leucine 436 and Methionine 439 of a peroxisome proliferator activated γ receptor.

111. The nuclear receptor modulator complex of claim 101, wherein the DHRS comprises residues Isoleucine 332, Leucine 356, Leucine 378 and Isoleucine 381 of a retinoic acid α receptor.

112. The nuclear receptor modulator complex of claim 101, wherein the DHRS comprises residues Isoleucine 346, Alanine 370, Methionine 394 and Leucine 397 of a pregnane X receptor.

113. The nuclear receptor modulator complex of claim 101, wherein the DHRS comprises residues Isoleucine 336, Serine 360, isoleucine 384 and Leucine 387 of a vitamin D receptor.

114. The nuclear receptor modulator complex of claim 101, wherein the at least one DHRS comprises residues Leucine 810, Isoleucine 835, Threonine 860, and Leucine 863 of an androgen receptor.

115. The nuclear receptor modulator complex of claim 101, wherein the at least one DHRS comprises residues Isoleucine 451, Threonine 483, Leucine 508, and Leucine 511 of an estrogen receptor.

116. The nuclear receptor modulator complex of claim 101, wherein the at least one DHRS comprises residues Leucine 824, Isoleucine 849, Threonine 874, and Leucine 877 of a progesterone receptor.

117. The nuclear receptor modulator complex of claim 101, wherein the nuclear receptor is selected from the group consisting of: a thyroid hormone receptor, a glucocorticoid receptor, an estrogen receptor, an androgen receptor, a mineralocorticoid receptor, a progestin receptor, a vitamin D receptor, a retinoid receptor, a retinoid X receptor, a peroxisomal proliferator activated receptor, an estrogen-receptor related receptor, a short heterodimer partner, a constitutive androstane receptor, a liver X receptor, a pregnane X receptor, a HNF-4 receptor, a farnesoid X receptor, and an orphan receptor.

118. The nuclear receptor modulator complex of claim 117, wherein the nuclear receptor comprises a nuclear receptor isoform.

119. The nuclear receptor modulator complex of claim 101, wherein the complex is in vitro.

120. The nuclear receptor modulator complex of claim 101, wherein the complex is in vivo.

121. The nuclear receptor modulator complex of claim 101, wherein the complex is in a cell.

122. The nuclear receptor modulator complex of claim 101, wherein the complex is in a mammal.

123. A library of modulators for a nuclear receptor, wherein the library comprises a plurality of different modulators that specifically bind a nuclear receptor dimer/heterodimer regulator site (DHRS) of a nuclear receptor.

124. The library of claim 123, wherein the library comprises between about 5 and 1000 members.

125. The library of claim 123, wherein the library comprises more than about 1000 members.

126. The library of claim 123, wherein the library comprises a phage display library.

127. A screening system for screening test agents that modulate dimer/heterodimer formation or cofactor molecule interaction of nuclear receptors, the screening system comprising:
at least one polypeptide, wherein the at least one polypeptide comprises a nuclear receptor dimer/heterodimer regulatory site (DHRS); and,
instructions for detecting dimer/heterodimerization or interactions of cofactor molecule of the at least one polypeptide.

128. The screening system of claim 127, wherein the at least one polypeptide comprise a full or partial nuclear receptor amino acid sequence.

129. The screening system of claim 127, wherein the at least one polypeptide is provided by a nucleic acid, wherein the nucleic acid encodes the at least one polypeptide.

130. A prescreening system for prescreening a test agent that bind to a nuclear receptor dimer/heterodimer regulator site (DHRS), the prescreening system comprising:
a polypeptide that comprises the DHRS; and,
instructions for detecting specific binding of the test agent to the DHRS.

131. A system for designing putative compounds that contact a nuclear receptor dimer/heterodimer regulatory site (DHRS), the system comprising:
a three dimensional model of a protein or polypeptide comprising a nuclear receptor dimer/heterodimer regulatory site (DHRS); and,
instructions for modeling binding of one or more compounds to the three dimensional model to design at least one putative compound that contacts the DHRS.
